(19) United States
(12) Patent Application Publication
(10) Pub. No.: US 2024/0336694 A1
(43) Pub. Date: Oct. 10, 2024

(54) COMPOSITIONS AND METHODS FOR AUGMENTING ANTIBODY MEDIATED RECEPTOR SIGNALING

(71) Applicant: Dana-Farber Cancer Institute, Inc., Boston, MA (US)

(72) Inventors: Wayne A. Marasco, Wellesley, MA (US); Quan Karen Zhu, Southborough, MA (US)

(21) Appl. No.: 18/528,382

(22) Filed: Dec. 4, 2023

Related U.S. Application Data
(62) Division of application No. 16/483,738, filed on Aug. 5, 2019, now abandoned.
(60) Provisional application No. 62/455,245, filed on Feb. 6, 2017.

(51) Int. Cl. C07K 16/28; A61P 35/02

(52) U.S. Cl. CPC C07K 16/2878; A61P 35/02; C07K 2317/524; C07K 2317/526; C07K 2317/53; C07K 2317/71; C07K 2317/732; C07K 2317/75

(57) ABSTRACT

The present invention provides compositions and methods for augmenting antibody mediate receptor signaling.

This application is a divisional of U.S. patent application Ser. No. 16/483,738, filed on Aug. 5, 2019, which is a U.S. National Stage Application of PCT Application No. PCT/US2018/017027 filed on Feb. 6, 2018, which claims the benefit of U.S. Provisional Patent Application No. 62/455,245 filed on Feb. 6, 2017, the entire disclosures of each of which are incorporated by reference in their entireties.

### SEQUENCE LISTING

The instant application contains a Sequence Listing which has been submitted electronically in ASCII format and is hereby incorporated by reference in its entirety. Said ASCII copy, created on Mar. 26, 2018, is named 5031461-002-WO1_SL.txt and is 79,185 bytes in size.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

The present invention relates generally to therapeutic antibodies with enhanced functions. Specifically, the invention concerns polypeptides comprising variants of an Fc region, and antibodies comprising the same. More particularly, the present invention concerns Fc region-containing polypeptides that have altered effector function as a consequence of one or more amino acid substitutions in the Fc region of the polypeptide.

### BACKGROUND OF THE INVENTION

Monoclonal antibodies have great therapeutic potential and play an important role in today's medical portfolio. During the last decade, a significant trend in the pharmaceutical industry has been the development of monoclonal antibodies (mAbs) as therapeutic agents for the treatment of a number of diseases, such as cancers, asthma, arthritis, and multiple sclerosis.

The Fc region of an antibody, i.e., the terminal ends of the heavy chains of antibody spanning domains CH2, CH3 and a portion of the hinge region, is limited in variability and is involved in effecting the physiological roles played by the antibody. The effector functions attributable to the Fc region of an antibody vary with the class and subclass of antibody and include binding of the antibody via the Fc region to a specific Fc receptor (“FcR”) on a cell, which triggers various biological responses.

### SUMMARY OF THE INVENTION

The invention features polypeptides comprising an Fc variant of a wild-type human IgG Fc region, for example, the Fc variant having amino acid substitutions E345K, E430G, L234A, and L235A; or E345K, E430G, S228P and R409K. The residues are numbered according to the EU index of Kabat (e.g., sec Edelman, et al., Proc Natl Acad Sci USA 63 (1969) 78-85). The polypeptide exhibits a reduced affinity to one or more of human Fc receptors and/or increased receptor clustering compared to the polypeptide having a wildtype IgG Fc region.

An aspect of the invention is directed to engineered polypeptides comprising an Fc variant of a wild-type human IgG Fc region. In one embodiment, the Fc variant comprises an amino acid substitution, or at least 2, 3, 4, 5, 6, or 7 substitutions, at residue positions 228, 234, 235, 345, 409, 430, 440, or a combination thereof, and wherein the amino acid residues are numbered according to the EU index of Kabat. In one embodiment, the amino acid at residue position 228 according to the EU index of Kabat is substituted with proline (P) or serine(S). In one embodiment, the amino acid at residue position 234 according to the EU index of Kabat is substituted with alanine (A). In one embodiment, the amino acid at residue position 235 according to the EU index of Kabat is substituted with alanine (A). In one embodiment, glutamate (E) at residue position 345 according to the EU index of Kabat is substituted with lysine (K), glutamine (Q), arginine (R), or tyrosine (Y). In one embodiment, the amino acid at residue position 409 according to the EU index of Kabat is substituted with lysine (K), or arginine (R). In one embodiment, glutamate (E) at residue position 430 according to the EU index of Kabat is substituted with glycine (G),serine(S), phenylalanine (F), or threonine (T). In one embodiment, serine(S) at residue position 440 according to the EU index of Kabat is substituted with tryptophan (W). In one embodiment, the amino acid substitutions comprise L234A, L235A, E345K, and E430G, and wherein the amino acid residues are numbered according to the EU index of Kabat. In one embodiment, the amino acid substitutions comprise S228P, E345K, R409K, and E430G, and wherein the amino acid residues are numbered according to the EU index of Kabat. In some embodiments, the polypeptide exhibits a reduced affinity to one or more of human Fc receptors compared to the polypeptide comprising the wildtype IgG Fc region. In other embodiments, the polypeptide further exhibits increased receptor clustering compared to the polypeptide comprising the wildtype IgG Fc region.

An aspect of the invention is directed to an engineered polypeptide comprising an Fc variant of a wild-type human IgG Fc region, wherein the Fc variant comprises an amino acid sequence comprising at least 90% identity to SEQ ID NO: 4, and wherein an amino acid substitution occurs at X₁, X₂, X₃, X₄, X₅, X₆, X₇, or a combination thereof. In one embodiment, the Fc variant comprises an amino acid sequence comprising at least 91%, at least 92%, at least 93%, at least 94%, at least 95%, at least 96%, at least 97%, at least 98%, at least 99%, or 100% identity to SEQ ID NO: 4. In one embodiment. X₁ is an amino acid substitution comprising serine(S). In one embodiment, X₂ is an amino acid substitution comprising alanine (A). In one embodiment, X₃ is an amino acid substitution comprising Alanine (A). In one embodiment, X₄ is an amino acid substitution comprising lysine (K), glutamine (Q). arginine (R), or tyrosine (Y). In one embodiment, X₅ is an amino acid substitution comprising lysine (K), or arginine (R). In one embodiment, X₆ is an amino acid substitution comprising glycine (G), serine(S), phenylalanine (F), or threonine (T). In one embodiment, X₇ is an amino acid substitution comprising tryptophan (W).fe

An aspect of the invention is directed to an engineered polypeptide comprising an Fc variant of a wild-type human IgG Fc region, wherein the Fc variant comprises an amino acid sequence comprising at least 90% identity to SEQ ID NO: 5, and wherein an amino acid substitution occurs at X₁, X₂, X₃, X₄, X₅, X₆, or a combination thereof. In one embodiment, the Fc variant comprises an amino acid sequence comprising at least 91%, at least 92%, at least 93%, at least 94%, at least 95%, at least 96%, at least 97%, at least 98%, at least 99%, or 100% identity to SEQ ID NO: 5. In one embodiment, X₁ is an amino acid substitution comprising serine(S). In one embodiment, X₂ is an amino acid substitution comprising alanine (A). In one embodiment, X₃ is an amino acid substitution comprising lysine (K), glutamine (Q), arginine (R), or tyrosine (Y). In one embodiment, X₄ is an amino acid substitution comprising lysine (K), or arginine (R). In one embodiment, X₅ is an amino acid substitution comprising glycine (G), serine(S), phenylalanine (F), or threonine (T). In one embodiment, X₆ is an amino acid substitution comprising tryptophan (W).

An aspect of the invention is directed to an engineered polypeptide comprising an Fc variant of a wild-type human IgG Fc region, wherein the Fc variant comprises an amino acid sequence comprising at least 90% identity to SEQ ID NO: 6, and wherein an amino acid substitution occurs at X₁, X₂, X₃, X₄, X₅, X₆, X₇, or a combination thereof. In one embodiment, the Fc variant comprises an amino acid sequence comprising at least 91%, at least 92%, at least 93%, at least 94%, at least 95%, at least 96%, at least 97%, at least 98%, at least 99%, or 100% identity to SEQ ID NO: 6. In one embodiment, X1 is a substitution of an amino acid at residue position 228 according to the EU index of Kabat and which comprises proline (P). In one embodiment, X₂ is an amino acid substitution comprising alanine (A). In one embodiment, X₃ is an amino acid substitution comprising Alanine (A). In one embodiment, X₄ is an amino acid substitution comprising lysine (K), glutamine (Q), arginine (R), or tyrosine (Y). In one embodiment, X₅ is an amino acid substitution comprising lysine (K), or arginine (R). In one embodiment, X₆ is an amino acid substitution comprising glycine (G), serine(S), phenylalanine (F), or threonine (T). In one embodiment, X₇ is an amino acid substitution comprising tryptophan (W).

The polypeptide is for example an antibody or an Fc fusion protein. The antibody is a monospecific antibody, bispecific antibody or multispecific antibody. The polypeptide can have a human IgG1, IgG2, or IgG4 Fc region. In some embodiments, the polypeptide can be antibody specific for an immune modulator, such as for example, CD27, OX40, 4-1BB, CD40L, ICOS and CD28. Optionally, polypeptide is an antibody specific for BCMA, CAIX, CCR4, PD-L1, PD-L2, PD1, Glucocorticoid-Induced Tumor Necrosis Factor Receptors (GITR), TIGIT, Severe acute respiratory syndrome (SARS), Middle East Respiratory Syndrome (MERS), influenza or flavivirus.

In one embodiment, the polypeptide is an antibody specific for Glucocorticoid-Induced Tumor Necrosis Factor Receptors (GITR). In one embodiment, the recombinant GITR antibody comprises the variable region amino acid sequences disclosed in Table 1B and the variant Fc region amino acid sequences disclosed in Table 3B (SEQ ID NOS: 18, 19, 21, 22, 24), Table 4B (SEQ ID NOS: 18, 19, 20, 22, 26). Table 5B (SEQ ID NOS: 18, 19, 22, 29, and 30), or Table 6B (SEQ ID NOS: 36, 37, 38, 40, and 42).

In one embodiment, the polypeptide is an antibody specific for CCR4. In one embodiment, the recombinant CCR4 antibody comprises the variable region amino acid sequences disclosed in Table 1B and the variant Fc region amino acid sequences disclosed in Table 3B (SEQ ID NOS: 18, 19, 21, 24), Table 4B (SEQ ID NOS: 18, 19, 20, 26). Table 5B (SEQ ID NOS: 18, 19, 29, and 30), Table 6B (SEQ ID NOS: 36, 37, 38, and 42), or SEQ ID NO: 44.

In various aspects, the polypeptide is conjugated to a drug, toxin, radiolabel, or a combination thereof as practiced in the art. In some embodiments, the toxin can be Pseudomonas exotoxin, ricin, botulinum toxin, or other toxins used by skilled artisans, such as those described by Polito et al (Biomedicines. 2016 Jun. 1: 4 (2), pii: E12. doi: 10.3390/biomedicines4020012) (which is incorporated by reference in its entirety). In some embodiments, the radiolabel can be Yttrium-90, Rhenium-188, Lutetium-177, strontium-89, radium-223, and the like. In some embodiments, the antibody drug conjugate can be monomethyl auirstatin E, or for example, others described by Schumacher et al., (J Clin Immunol. 2016 May; 36 Suppl 1:100-7. doi: 10.1007/s10875-016-0265-6. Epub 2016 Mar. 22) (which is incorporated by reference in its entirety).

Also included in the invention are methods of treating a subject afflicted with a disease by administering a polypeptide according to the invention, or nucleic acid encoding the same. Also included in the invention are methods of treating a subject afflicted with a disease by administering to the subject a therapeutically effective amount of a composition comprising a polypeptide according to the invention or a nucleic acid encoding the same and a pharmaceutically acceptable carrier. In one embodiment, the invention provides for methods of treating a tumor in a subject wherein the method comprises administering to the subject the recombinant GITR antibody described herein. In one embodiment, the tumor is a solid tumor or liquid tumor. In some embodiments, the liquid tumor can be multiple myeloma, Acute myeloid leukemia (AML), or Acute lymphoblastic leukemia (ALL). In one embodiment, the invention provides for treating a blood-based cancer in a subject wherein the method comprising administering to a subject the recombinant CCR4 antibody described herein. In one embodiment, the blood-based cancer is a lymphoma or a leukemia.

In other aspects, the invention provides methods of enhancing cellular signaling or inducing receptor clustering of a cell by contacting the cell with an antibody capable of binding a ligand on the cell comprising an Fc variant of a wild-type human IgG Fc region. The Fc variant has an amino acid substitution, such as an amino acid substitution at E345, E430 and/or S440 wherein the residues are numbered according to the EU index of Kabat. In one embodiment, mutations include one or more of E430G, E430S, E430F, E430T, E345K, E345Q, E345R, E345Y, or S440W.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1. SDS-PAGE analysis of anti-GITR antibodies expressed and purified from 293F cells, pTCAE plasmids encoding anti-GITR antibody E1-3H7 IgG1 LALA (lane 1), E1-3H7 stabilized IgG4 (lane 2), CTI-10 stabilized IgG4 (lane 3), E1-3H7 IgG1 LALA hexamer (lane 4), E1-3H7 stabilized IgG4 hexamer (lane 5), and E1-3H7 IgG1 WT hexamer (lane 6) are transiently transfected into 293F cells. Cell supernatants were harvested 96 hours later and purified with Protein A affinity resins. Circa 2 ug (as determined by OD280 reading post-purification) of each purified antibodies were analyzed by 4-20% polyacrylamide gel and visualized by Coomassie Blue staining. Lane 7 contains control CTI-10 IgG1 with known concentration. Panel A Reducing Condition; panel B non-reducing condition. Data show that each antibody were expressed and purified.

FIG. 2 is an illustration showing GITR-GITRL interaction activates the NF-KB pathway within the GloResponse NF-KB-luc2P/GITR Jurkat cell assay system made by Promega and used in our assays.

FIG. 3. The GloResponse NF-KB-luc2P/GITR Jurkat cells are reporter cells that produce luciferase activity based ligand or antibody reaction with surface expressed receptor GITR. As system controls, panel A shows that GITR ligand (GITRL) induced luciferase activity as expected and panel B presents the data that anti-HA antibody further enhances luciferase activity induced with 111 ng/ml GITRL (Note that GLTRL is fused with a c-terminal HA-tag). Panel C shows that our newly discovered anti-GITR antibody E1-3H7-sIgG4 can induce GiTR/NF-KB dependent luciferase alone or further enhances luciferase activity induced with 111 ng/ml GITRL, which is different from the behavior of a commercial anti-GITR Ab control, CTI-10, Panel D.

FIG. 4. Hexamerized anti-GITR E1-3H7 antibodies have increased sensitivity in mediating GITR/NF-KB dependent luciferase activities. (A) Anti-GITR antibody E1-3H7 IgG1-LALA and corresponding hexamer (E1-3H7-LALA Hex) induced luciferase activities in a dosage-dependent manner from the GloResponse NF-KB-luc2P/GITR Jurkat cells.

Note that E1-3H7 hexamers were capable to shift the luciferase induction to roughly 1 log lower in antibody concentration. (B) Anti-GITR E1-3H7 antibodies further potentiate GITRL induced luciferase activity. Once again, E1-3H7-LALA hexamers accomplished such induction at much lower Ab concentration. Panels C & D show that similar effects with E1-3H7 stabilized IgG4 and its corresponding sIgG4 hexamer. Anti-GITR E1-3H7 antibodies were used in a 3-fold dilution from 5000 ng/ml to 20.58 ng/ml in the absence (Panels A & C) or presence (Panels B & D) of 111 ng/ml GITR ligand.

FIG. 5. Hexamerized anti-GITR E1-3H7 antibodies have increased sensitivity in mediating GITR/NF-KB dependent luciferase activities. A similar experiment as shown in FIG. 4 except that in order to see the full extent of luciferase inductions, anti-GITR E1-3H7-IgG1 LALA or IgG4 antibody concentrations were used in a 3-fold dilution from 15000 ng/ml to 61.73 ng/ml in the absence (Panels A & C) or presence (Panels B & D) of 111 ng/ml GITR ligand while their corresponding hexamer formats remained at 5000 ng/ml to 20.58 ng/ml. An irrelevant IgG control showed no significant effect on the base level of luciferase induction by 111 ng/ml of GITRL.

FIG. 6. IgG1 Fc wild type, IgG1 Fc LALA mutant or stabilized IgG4 hexamers of anti-GITR E1-3H7 antibodies have similar activities in mediating GITR/NF-KB dependent luciferase activities. Anti-GITR E1-3H7-IgG1 WT or IgG1 LALA or sIgG4 hexamer antibody concentrations were used in a 3-fold dilution from 5000 ng/ml to 20.58 ng/ml in the absence or presence of 111 ng/ml GITR ligand while a control IgG1 has a concentration from 15000 ng/ml to 61.73 ng/ml. Note that E1-3H7 IgG1 WT hexamer results in Panel A were from a separate experiment than those presented in panels B & Cor panels D & E. The X and Y axis are the same for Panels A-E.

FIG. 7. ADCC assays using a reporter system from Promega.

FIG. 8. Nucleic acid and amino acid sequence of Fc regions of WT (SEQ ID NOS 72-73, respectively, in order of appearance) and LALA hexamer mutants (SEQ ID NOS 74-75, respectively, in order of appearance) of IgG1.

FIG. 9. Nucleic acid and amino acid sequence of Fc regions of stabilized hexamer IgG4 (SEQ ID NOS 76-77, respectively, in order of appearance).

FIG. 10. Expression vector map for vector that can be used for mammalian expression of IgG antibodies.

FIG. 11. Expression vector map for vector that can be used for mammalian expression of IgG antibodies.

FIG. 12. Amino acid sequence (SEQ ID NO: 1) for a wild type Fc region of IgG1 and the corresponding amino acid residue number according to the EU index of Kabat. FIG. 12 discloses residues 1-98, 114-223, 224-330, and 99-113 of SEQ ID NO: 1.

FIG. 13. Amino acid sequence (SEQ ID NO: 2) for a wild type Fc region of IgG2 and the corresponding amino acid residue number according to the EU index of Kabat. FIG. 13 discloses residues 1-98, 111-219, 220-326, and 99-110 of SEQ ID NO: 2.

FIG. 14. Amino acid sequence (SEQ ID NO: 3) for a wild type Fc region of IgG4 and the corresponding amino acid residue number according to the EU index of Kabat. FIG. 14 discloses residues 1-98, 111-220, 221-327, and 99-110 of SEQ ID NO: 3.

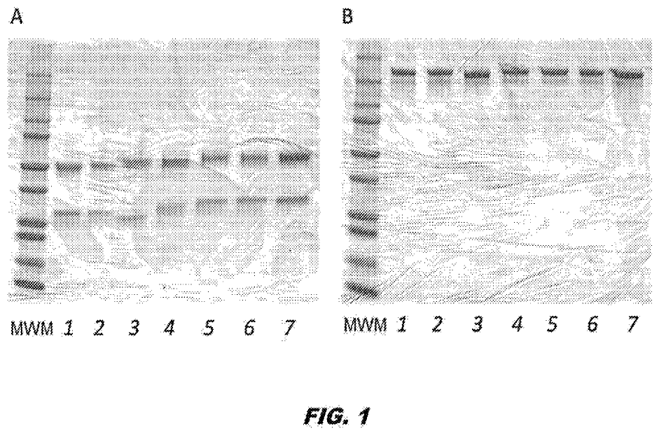

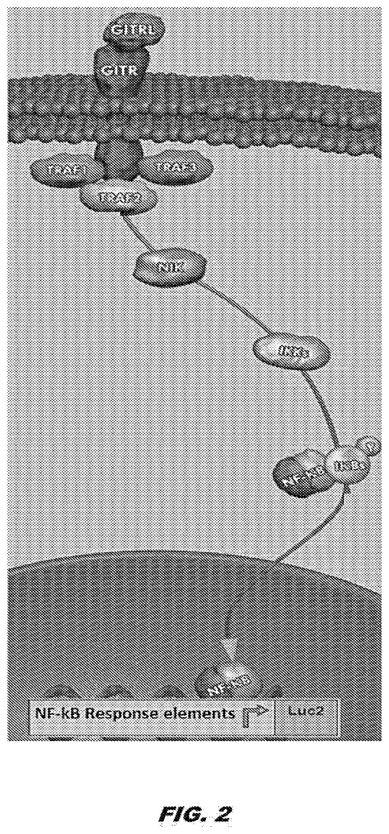

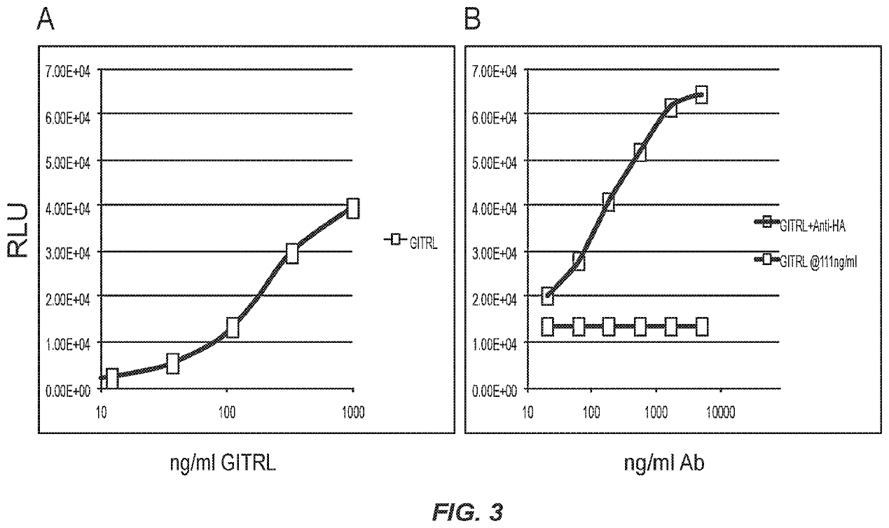

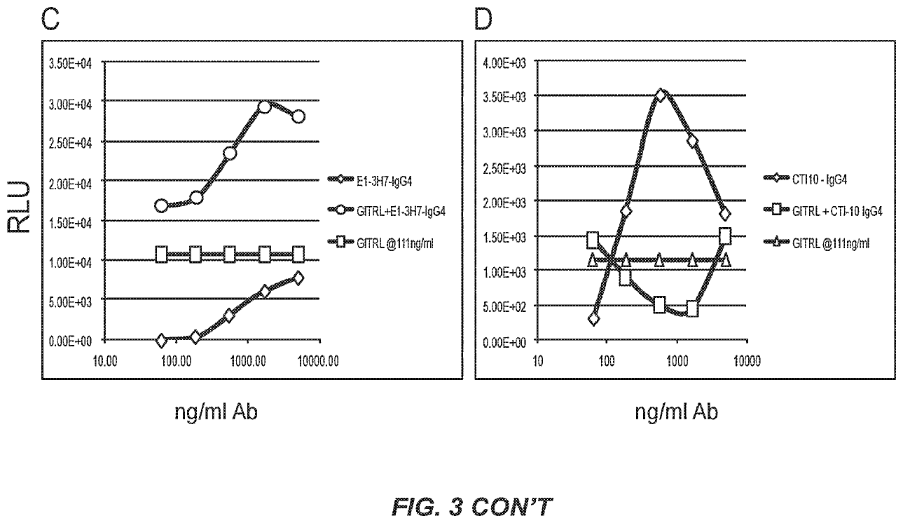

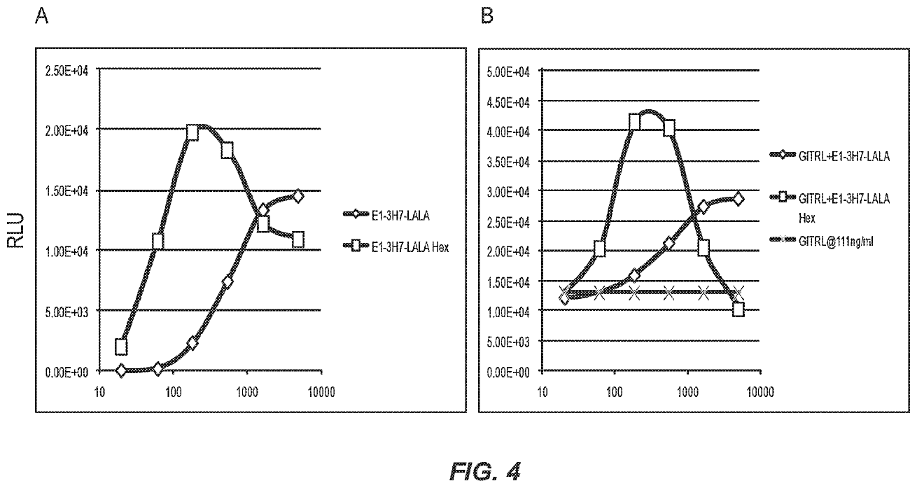

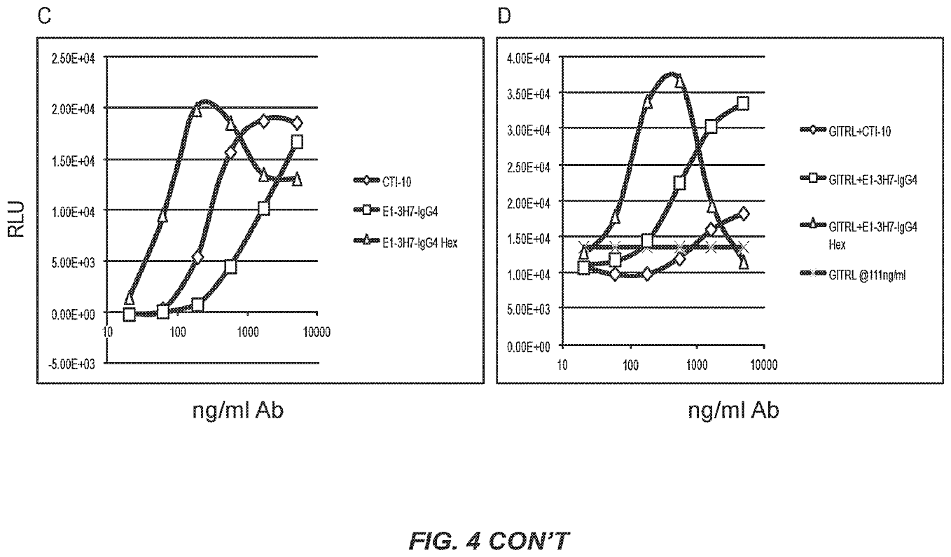

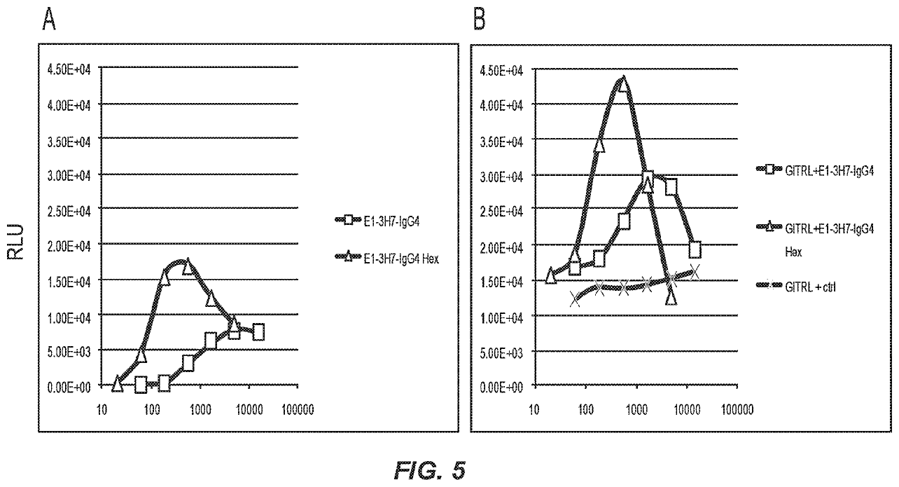

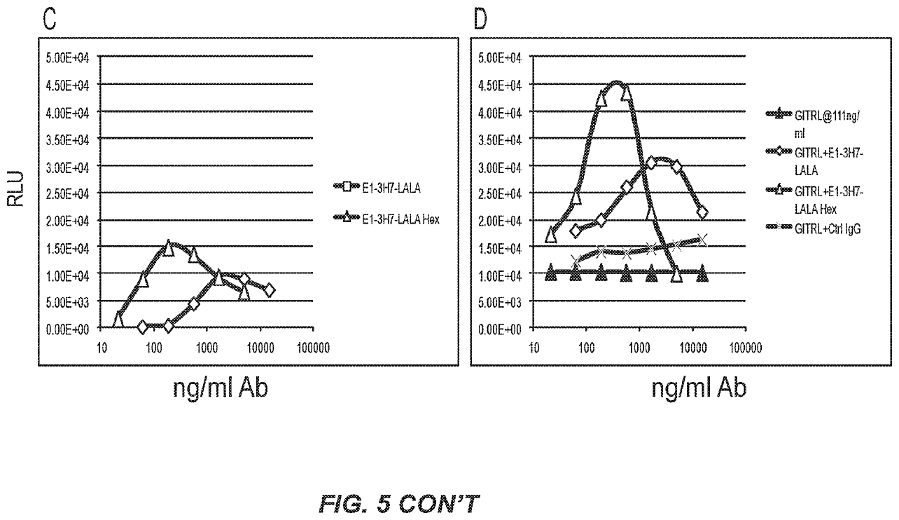

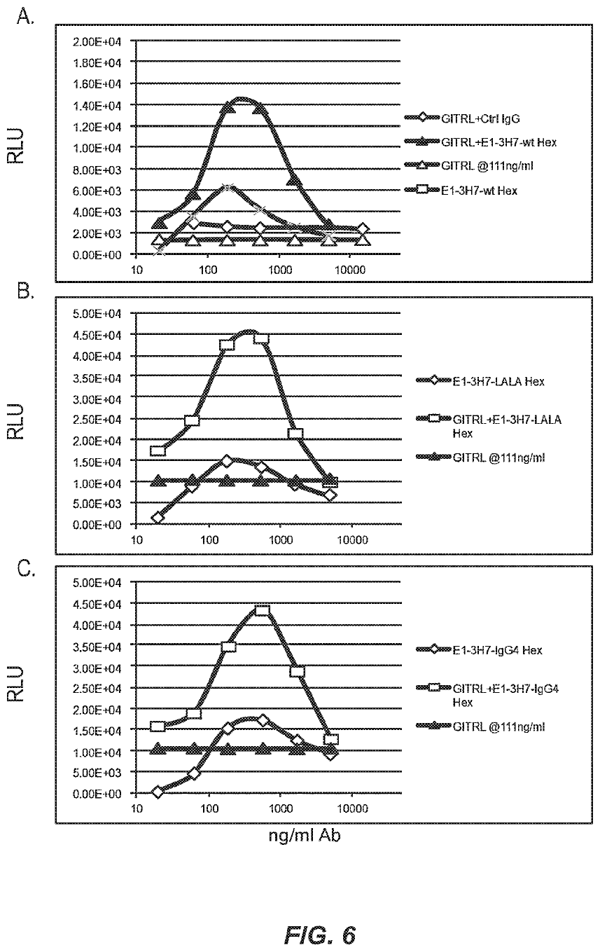

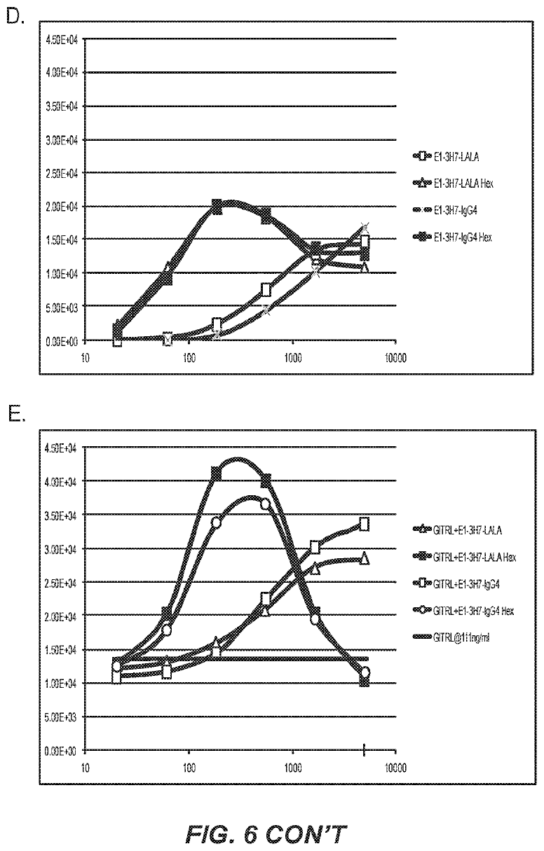

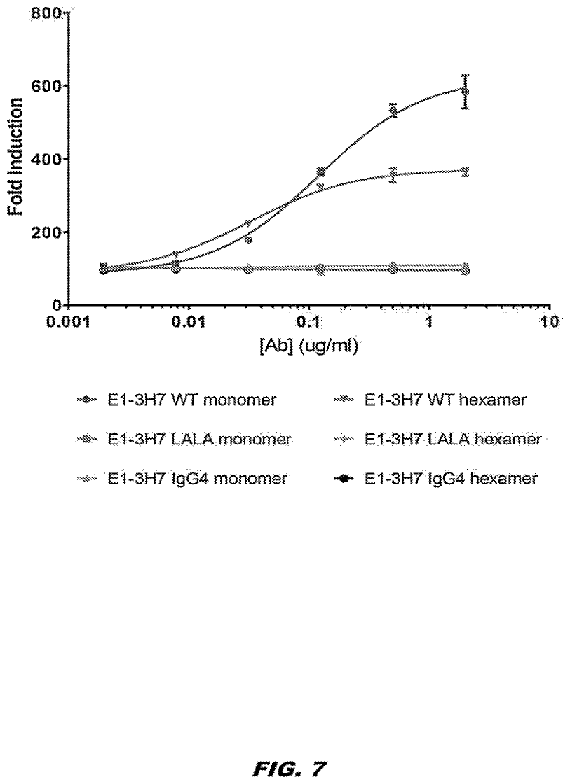

### DETAILED DESCRIPTION OF THE INVENTION

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

Fc receptors can have an extracellular domain that mediates binding to Fc, a membrane-spanning region, and an intracellular domain that may mediate some signaling event within the cell. These receptors are expressed in a variety of immune cells including monocytes, macrophages, neutrophils, dendritic cells, cosinophils, mast cells, platelets, B cells, large granular lymphocytes, Langerhans' cells, natural killer (NK) cells, and T cells. Formation of the Fc/FcγR complex recruits these effector cells to sites of bound antigen, typically resulting in signaling events within the cells and important subsequent immune responses such as release of inflammation mediators, B cell activation, endocytosis, phagocytosis, and cytotoxic attack.

In many circumstances, the binding and stimulation of effector functions mediated by the Fc region of immunoglobulins is highly beneficial, e.g. for a CD20 antibody, however, in certain instances it can be more advantageous to decrease or even to eliminate the effector function.

In other instances, for example, where blocking the interaction of a widely expressed receptor with its cognate ligand is the objective, it would be advantageous to decrease or eliminate all antibody effector function to reduce unwanted toxicity. It would also be advantageous to enhance signaling by increasing receptor clustering.

There is an unmet need for antibodies with a strongly decreased effector function such as ADCC and/or ADCP and/or CDC and enhanced receptor cell signaling and/or inducing receptor cell clustering. Therefore, the aim of the current invention was to synthesize and/or engineer polypeptides of the Fc region of immunoglobulins with mutations introduced to precipitate such effects and ultimately identify antibodies comprising the engineered Fc regions.

The invention is based in part upon the discovery that mutations in the Fc region of antibodies known to promote antibody hexamerization and increased complement-dependent cytotoxicity (CDC) also has the unexpected ability to markedly enhance effector cell signaling. The polypeptide variants, including the antibody variants, of the invention all comprise a binding region and a full-length or partial Fc domain of an immunoglobulin comprising one or more mutation(s) known to promote antibody hexamerization and reduced effector function.

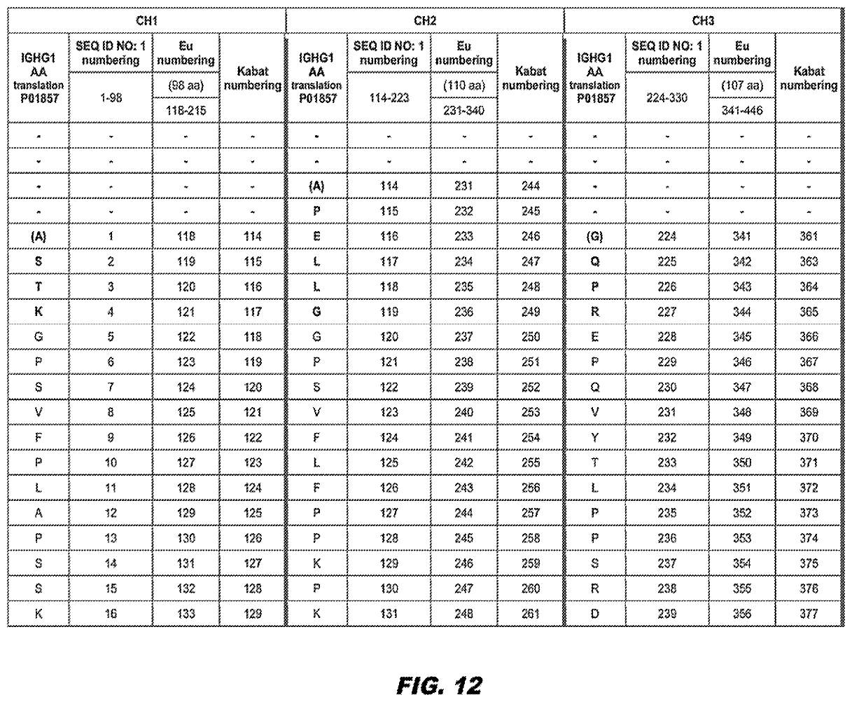

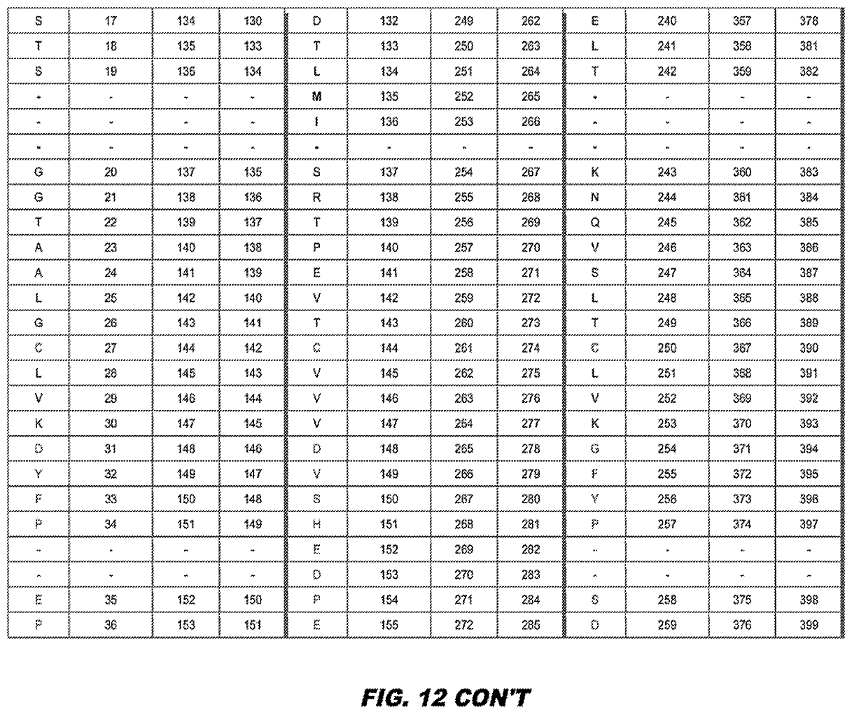

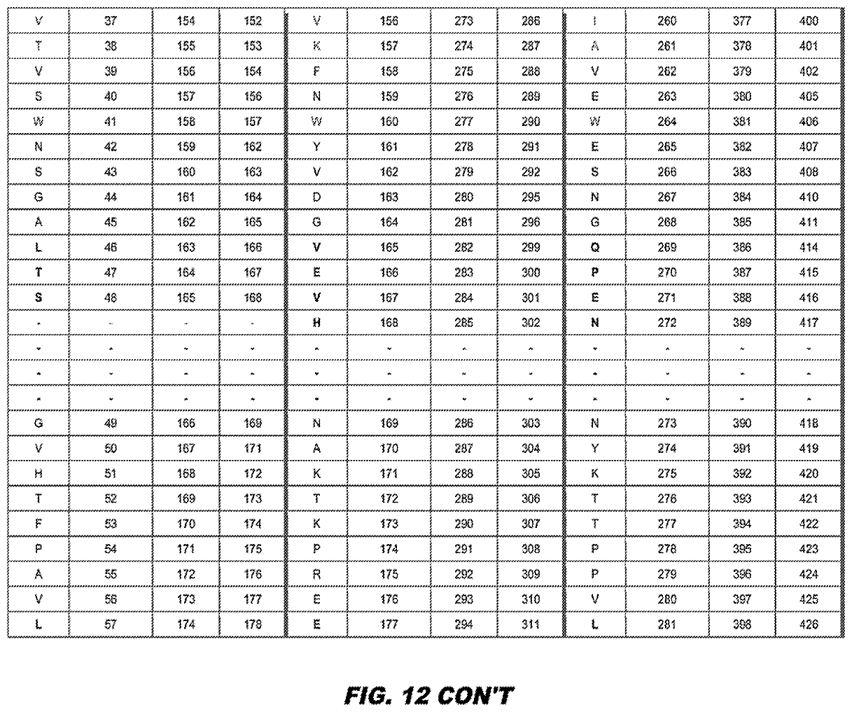

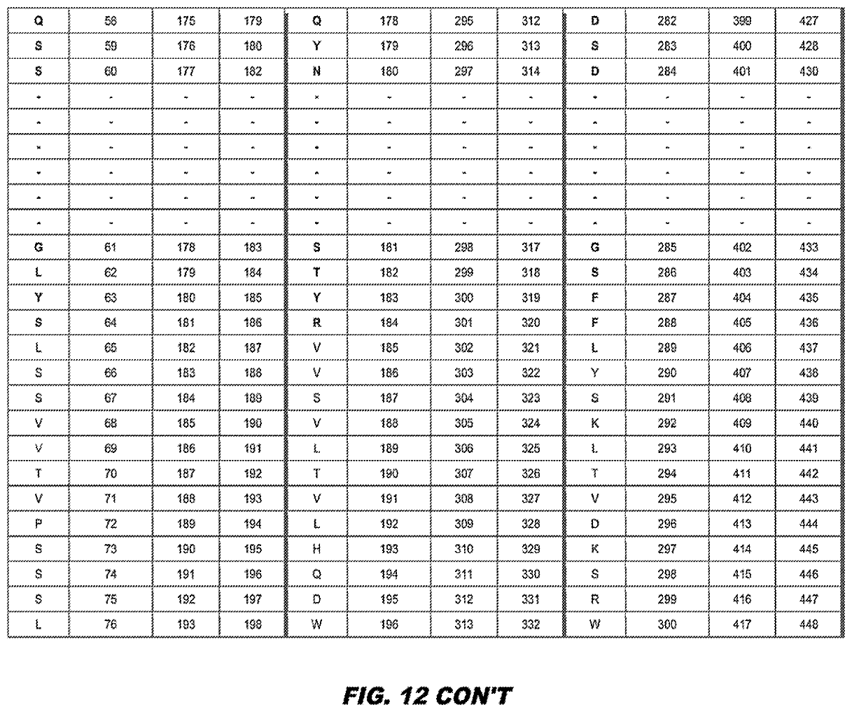

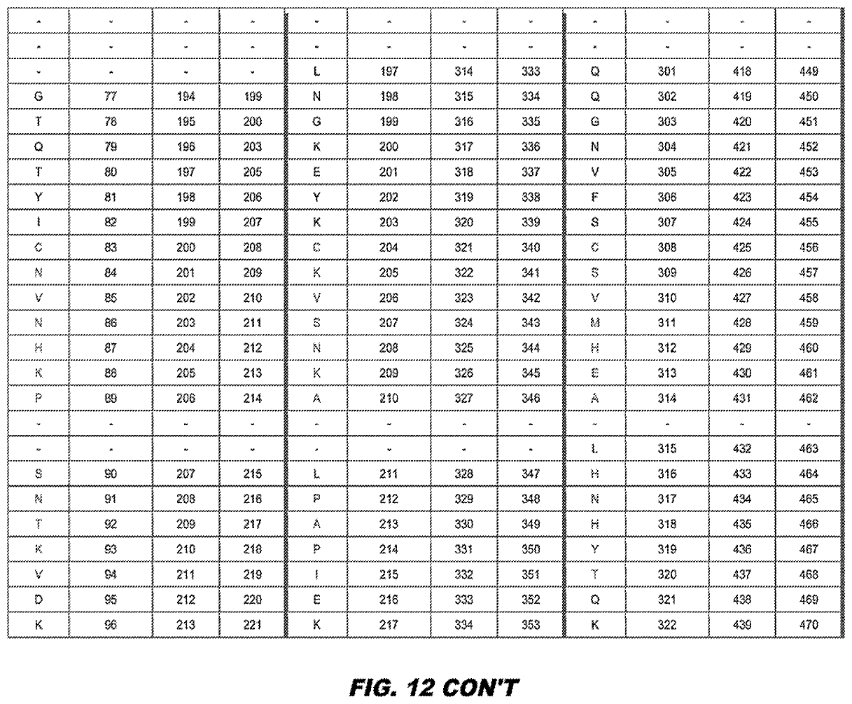

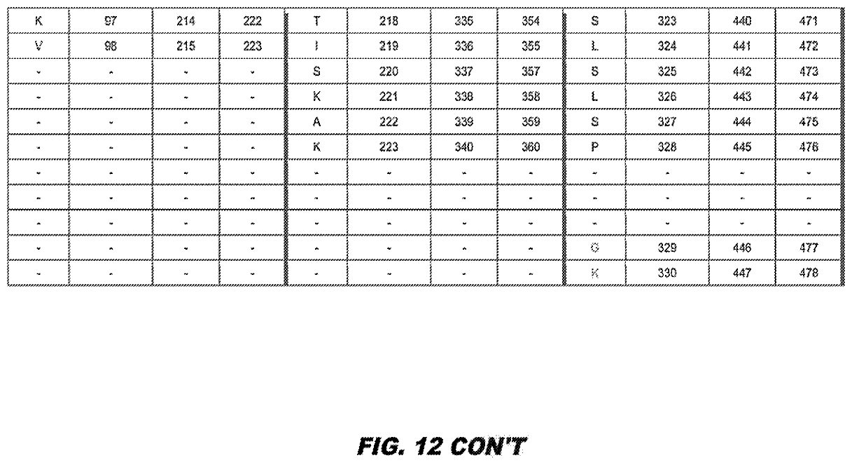

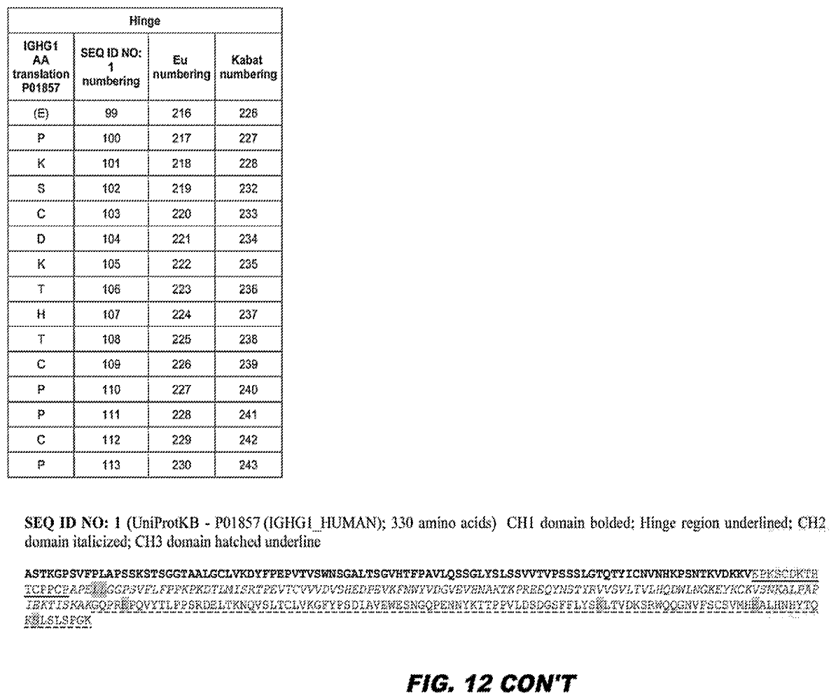

SEQ ID NO: 1 provides for the amino acid sequence of the wildtype Fc region of IgG1 (UniProtKB-P01857 (IGHG1_HUMAN); 330 amino acids), where the CH1 domain is bolded; the Hinge region is underlined; the CH2 domain italicized; the CH3 domain is hatched underlined; the shadowed boxes are amino acids that could be substituted according to the invention. FIG. 12 is a table that corresponds SEQ ID NO: 1 with the amino acid residues that are numbered according to the EU index of Kabat.

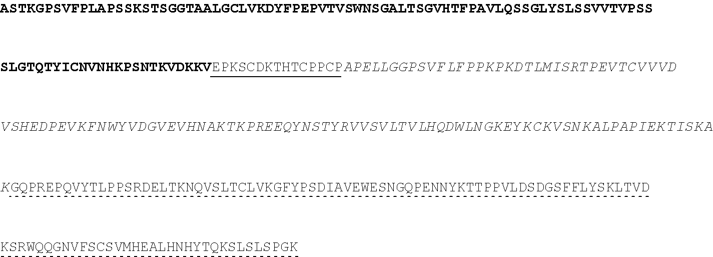

SEQ ID NO: 4 provides for the amino acid sequence of the variant Fc region of IgG1 (UniProtKB-P01857 (IGHG1_HUMAN); 330 amino acids), where the CH1 domain is bolded; the Hinge region is underlined; the CH2 domain italicized; the CH3 domain is hatched underlined; the shadowed boxes represent the amino acid residues that could be substituted according to the invention, wherein X1 is a substitution of an amino acid at residue position 228 according to the EU index of Kabat and which comprises proline (P); X₂ is a substitution of an amino acid at residue position 234 according to the EU index of Kabat and which comprises alanine (A); X₃ is a substitution of an amino acid at residue position 235 according to the EU index of Kabat and which comprises Alanine (A); X₄ is a substitution of an amino acid at residue position 345 according to the EU index of Kabat and which comprises lysine (K), glutamine (Q), arginine (R), or tyrosine (Y); X₅ is a substitution of an amino acid at residue position 409 according to the EU index of Kabat and which comprises arginine (R); X₆ is a substitution of an amino acid at residue position 430 according to the EU index of Kabat and which comprises glycine (G), serine (S), phenylalanine (F), or threonine (T); and X₇ is a substitution of an amino acid at residue position 440 according to the EU index of Kabat and which comprises tryptophan (W).

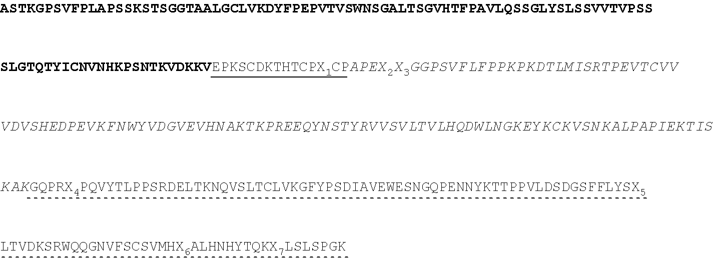

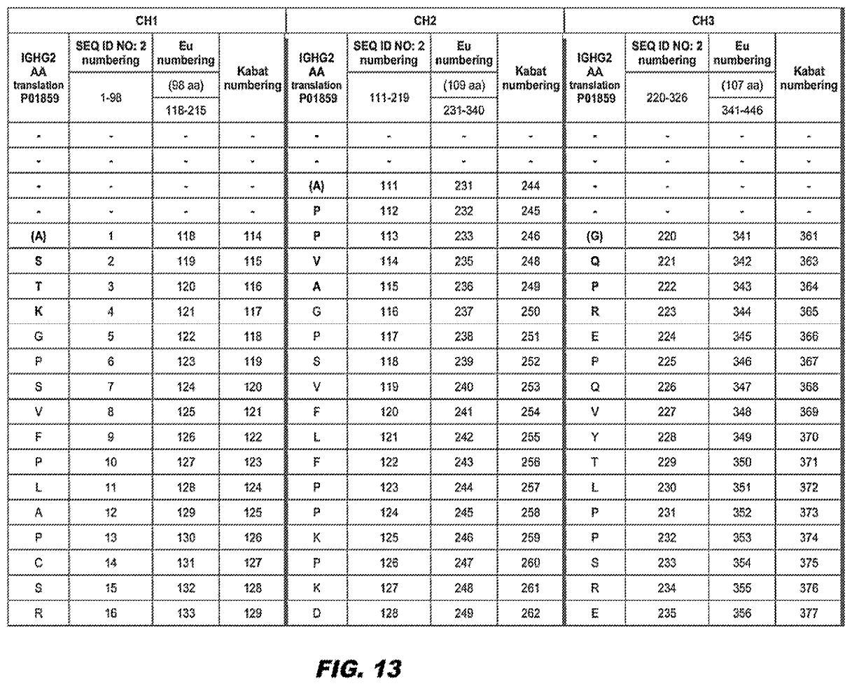

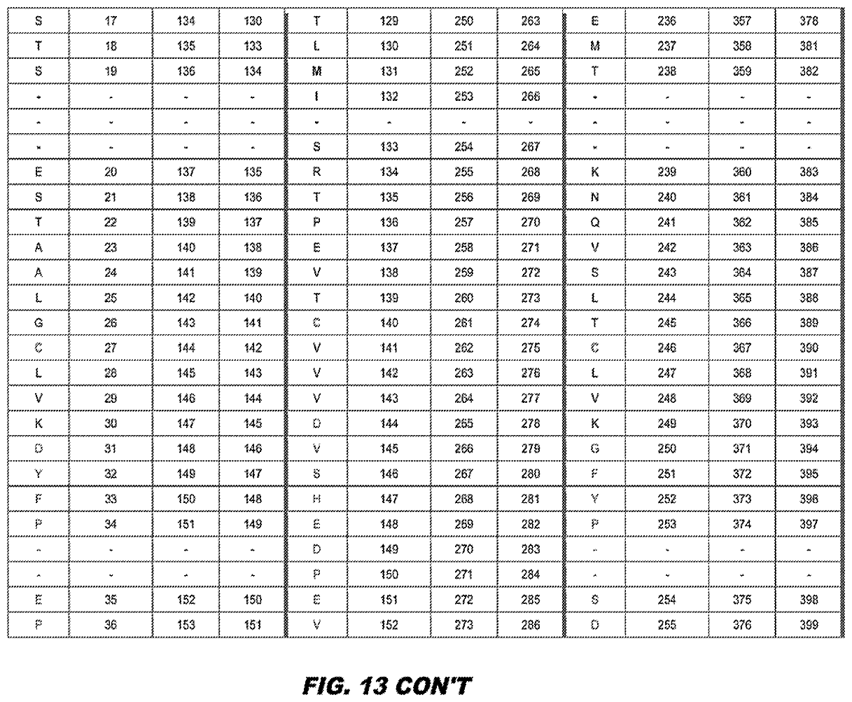

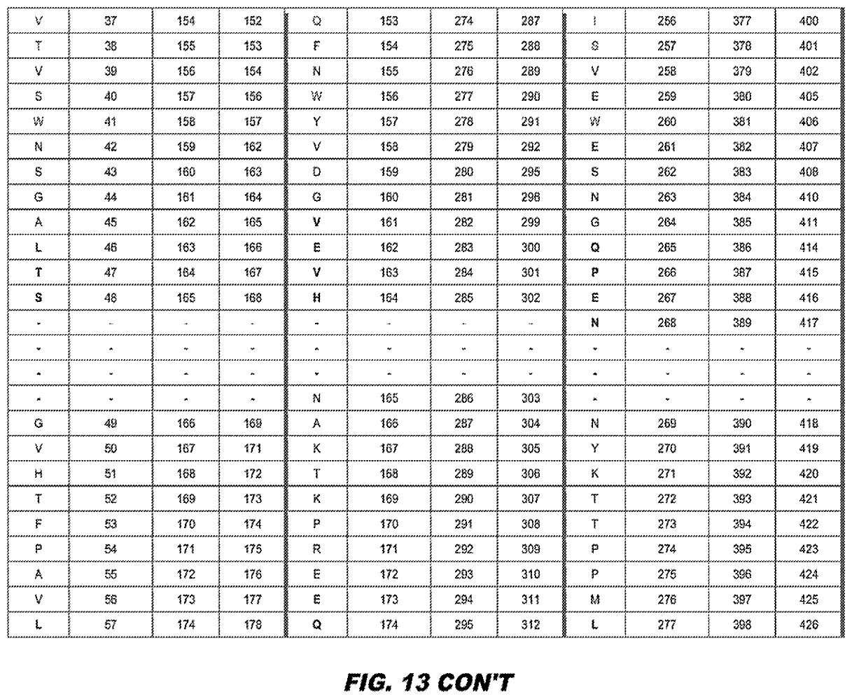

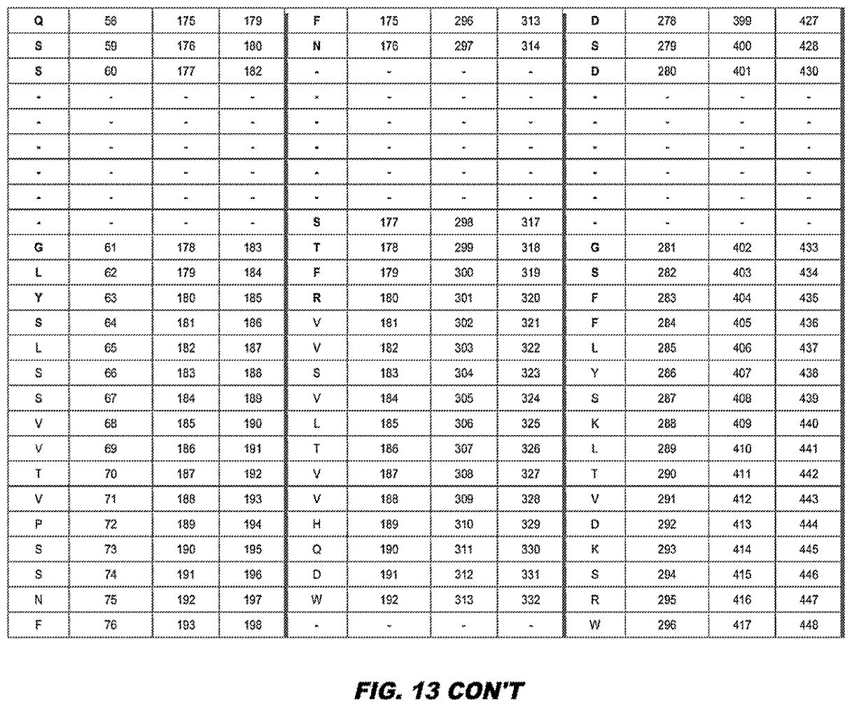

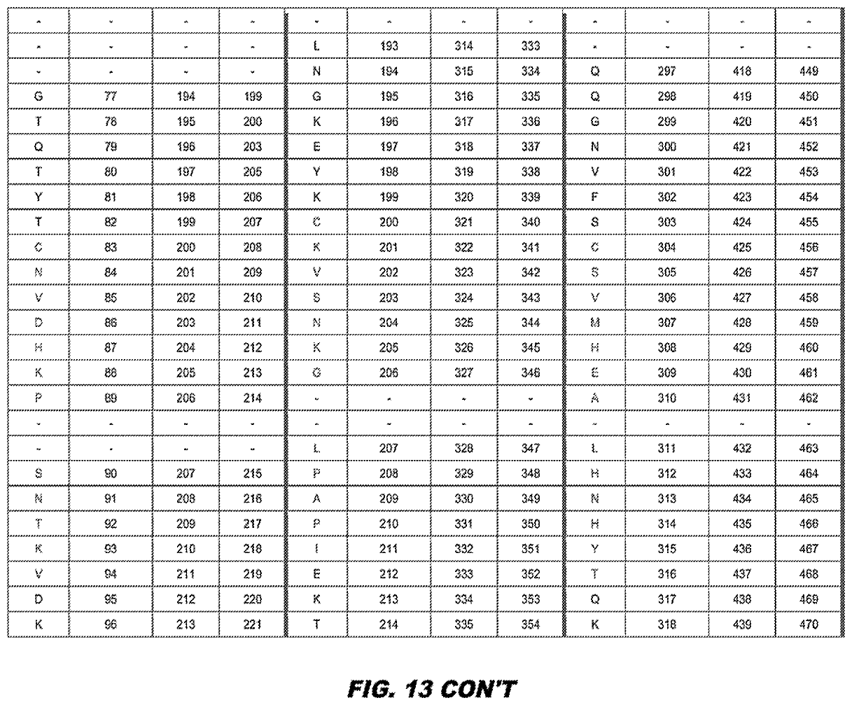

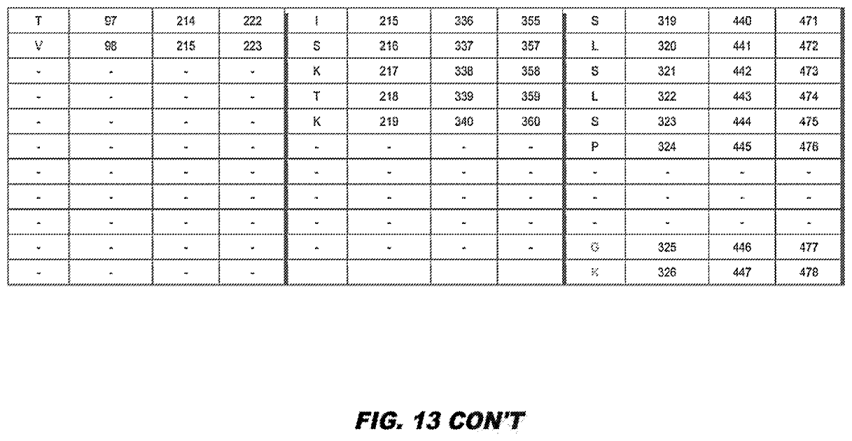

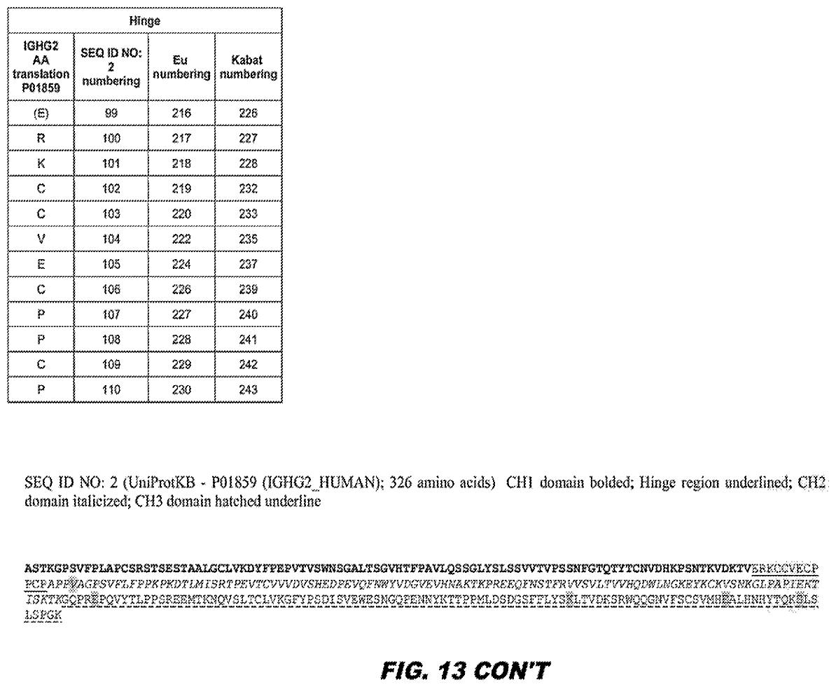

SEQ ID NO: 2 provides for the amino acid sequence of the wildtype Fc region of IgG2 (UniProtKB-P01859 (IGHG2_HUMAN); 326 amino acids), where the CH1 domain is bolded; the Hinge region is underlined; the CH2 domain italicized; the CH3 domain is hatched underlined; the shadowed boxes are amino acids that could be substituted according to the invention. FIG. 13 is a table that corresponds SEQ ID NO: 2 with the amino acid residues that are numbered according to the EU index of Kabat.

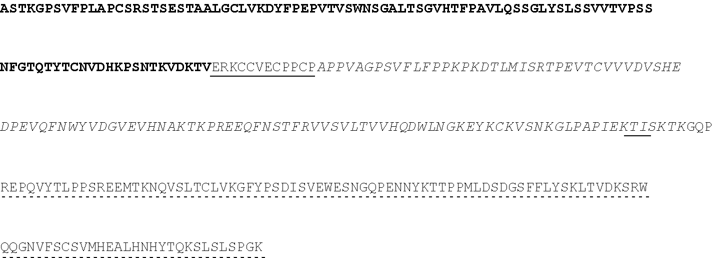

SEQ ID NO: 5 provides for the amino acid sequence of the variant Fc region of IgG2 (UniProtKB-P01859 (IGHG2_HUMAN); 326 amino acids), where the CH1 domain is bolded; the Hinge region is underlined; the CH2 domain italicized; the CH3 domain is hatched underlined; the shadowed boxes represent the amino acid residues that could be substituted according to the invention, wherein X1 is a substitution of an amino acid at residue position 228 according to the EU index of Kabat and which comprises proline (P); X₂ is a substitution of an amino acid at residue position 235 according to the EU index of Kabat and which comprises alanine (A); X₃ is a substitution of an amino acid at residue position 345 according to the EU index of Kabat and which comprises lysine (K), glutamine (Q), arginine (R), or tyrosine (Y); X₄ is a substitution of an amino acid at residue position 409 according to the EU index of Kabat and which comprises arginine (R); X₅ is a substitution of an amino acid at residue position 430 according to the EU index of Kabat and which comprises glycine (G), serine(S), phenylalanine (F), or threonine (T); and X₆ is a substitution of an amino acid at residue position 440 according to the EU index of Kabat and which comprises tryptophan (W).

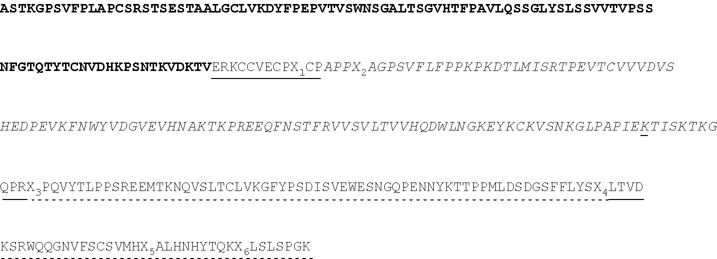

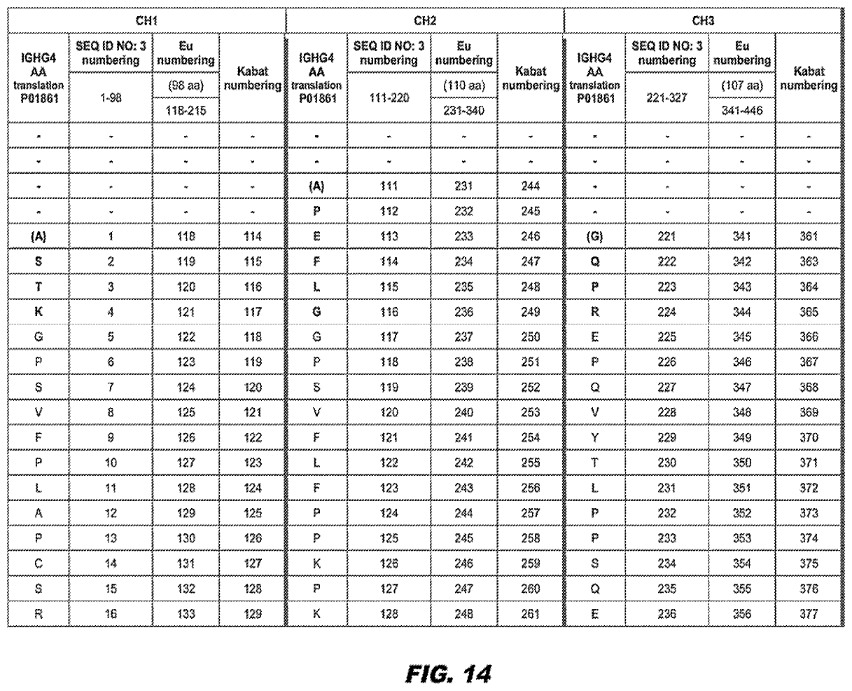

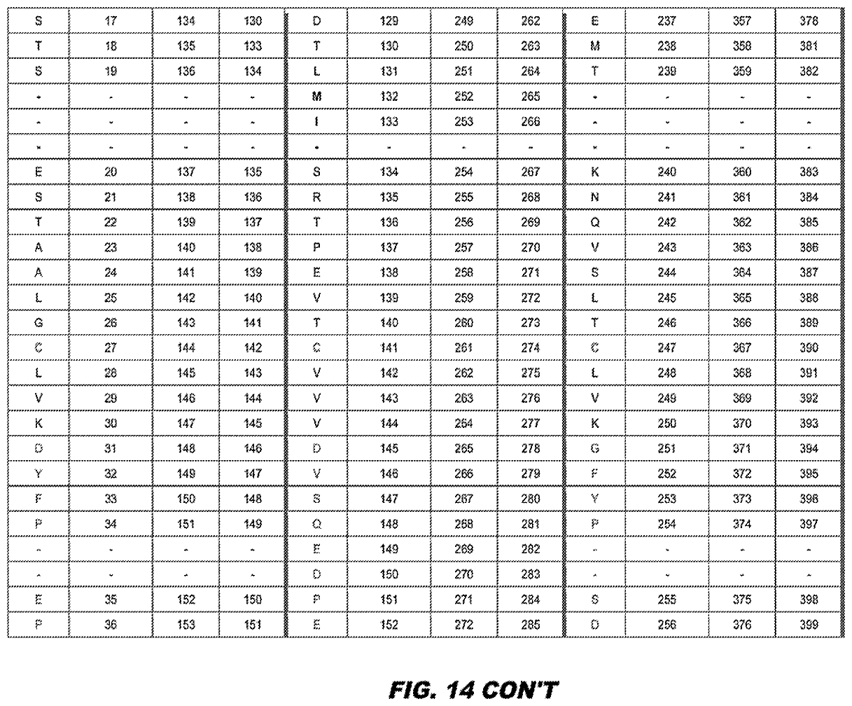

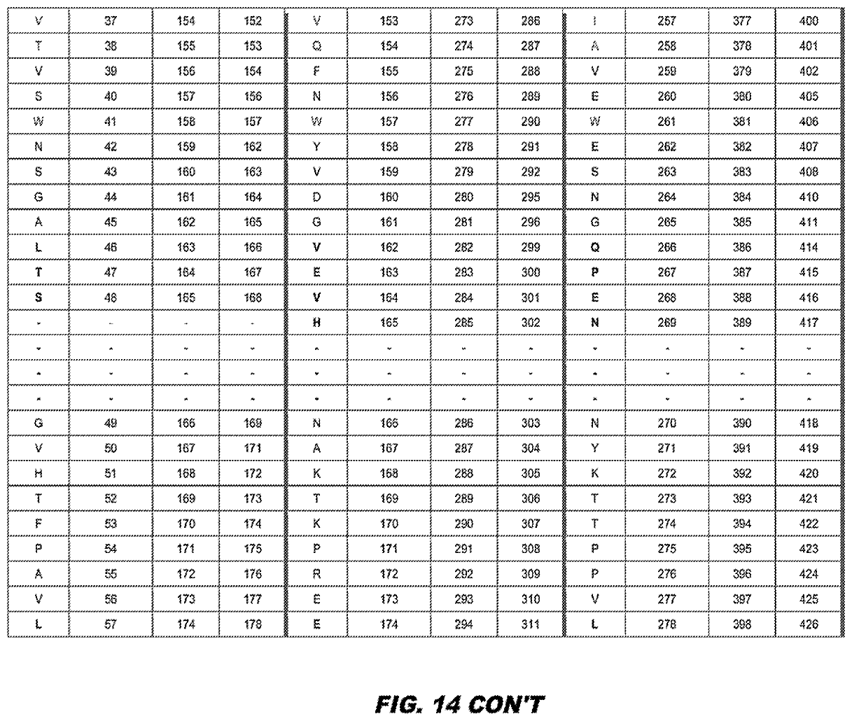

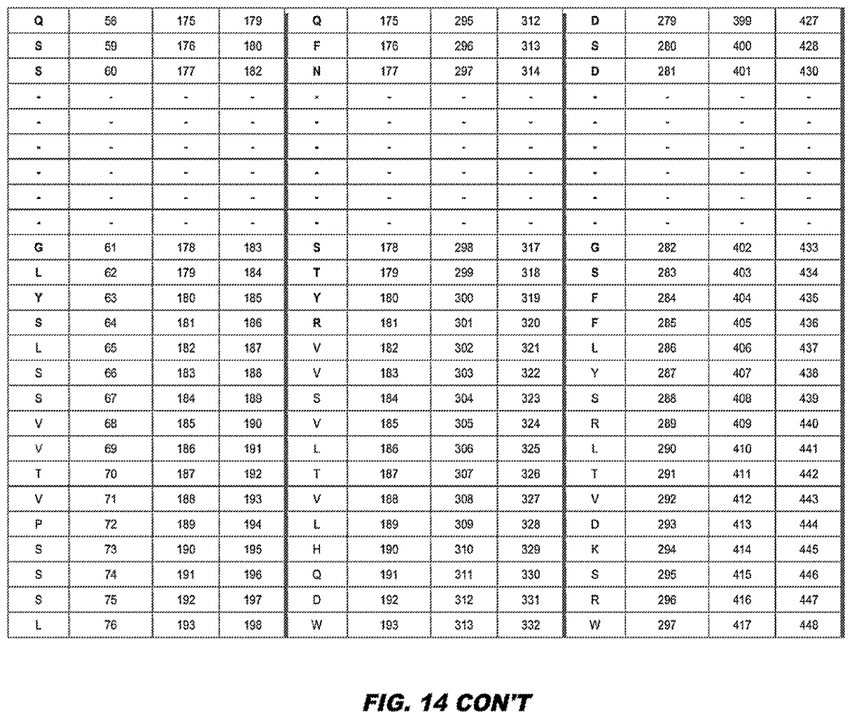

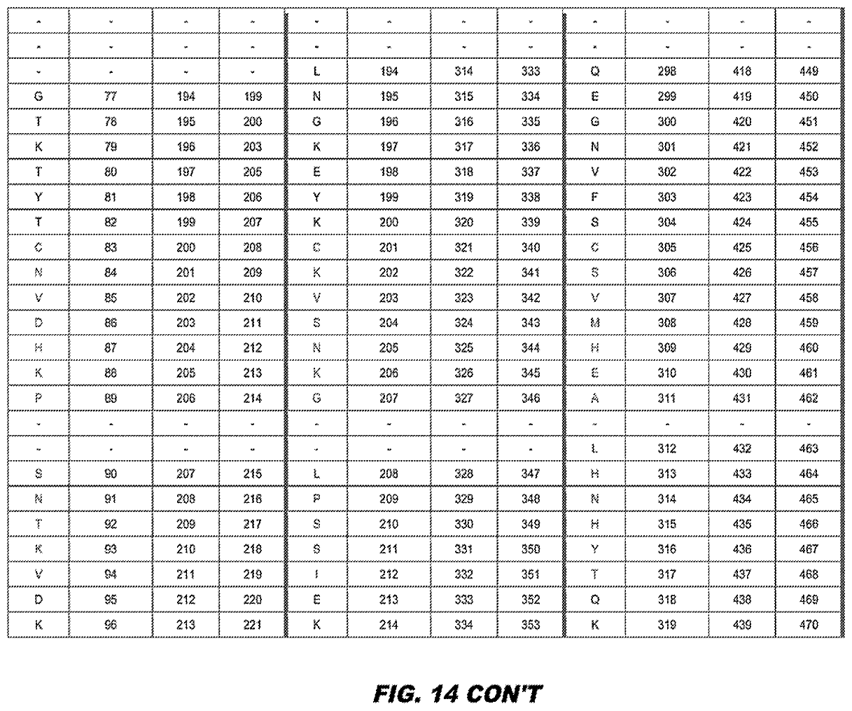

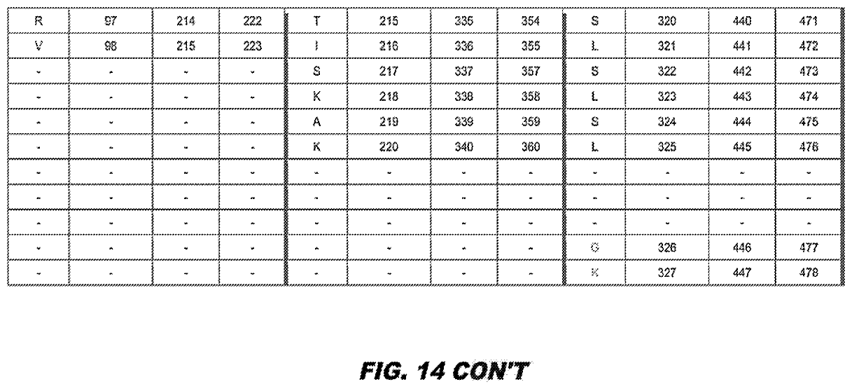

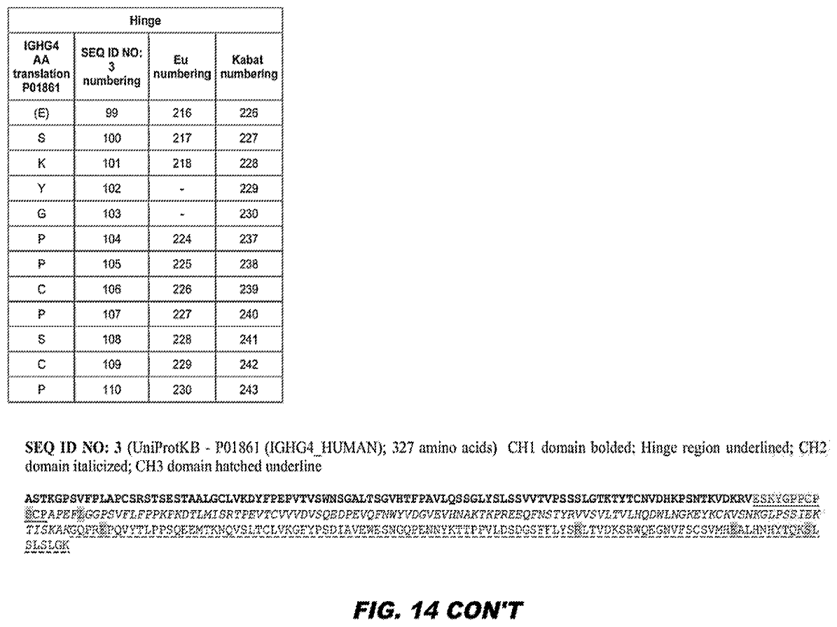

SEQ ID NO: 3 provides for the amino acid sequence of the wildtype Fc region of IgG4 (UniProtKB-P01861 (IGHG4_HUMAN); 327 amino acids), where the CH1 domain is bolded; the Hinge region is underlined; the CH2 domain italicized; the CH3 domain is hatched underlined; the shadowed boxes are amino acids that could be substituted according to the invention. FIG. 14 is a table that corresponds SEQ ID NO: 3 with the amino acid residues that are numbered according to the EU index of Kabat.

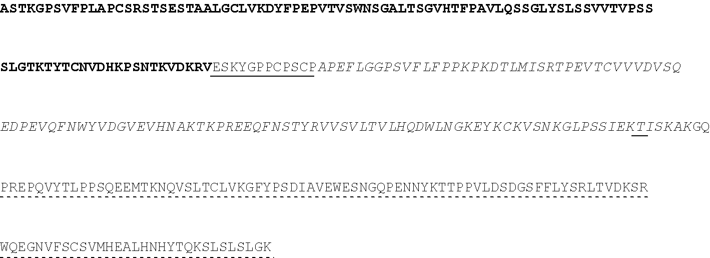

SEQ ID NO: 6 provides for the amino acid sequence of the variant Fc region of IgG4 (UniProtKB-P01861 (IGHG4_HUMAN); 327 amino acids), where the CH1 domain is bolded; the Hinge region is underlined; the CH2 domain italicized; the CH3 domain is hatched underlined; the shadowed boxes represent the amino acid residues that could be substituted according to the invention, wherein X₁ is a substitution of an amino acid at residue position 228 according to the EU index of Kabat and which comprises proline (P); X₂ is a substitution of an amino acid at residue position 234 according to the EU index of Kabat and which comprises alanine (A); X₃ is a substitution of an amino acid at residue position 235 according to the EU index of Kabat and which comprises Alanine (A); X₄ is a substitution of an amino acid at residue position 345 according to the EU index of Kabat and which comprises lysine (K), glutamine (Q), arginine (R), or tyrosine (Y); X₅ is a substitution of an amino acid at residue position 409 according to the EU index of Kabat and which comprises lysine (K); X₆ is a substitution of an amino acid at residue position 430 according to the EU index of Kabat and which comprises glycine (G), serine (S), phenylalanine (F), or threonine (T); and X₇ is a substitution of an amino acid at residue position 440 according to the EU index of Kabat and which comprises tryptophan (W).

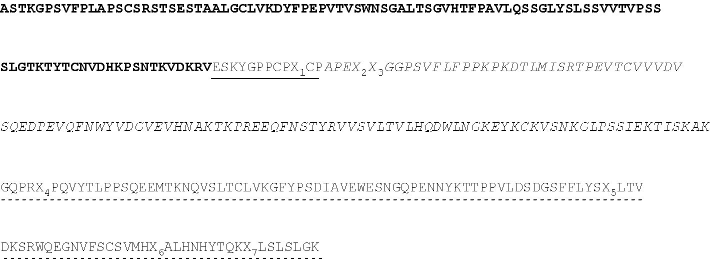

Fc mutations that can promote antibody hexamerization include one or more mutation(s) in the segment corresponding to amino acid residues at about positions 345 to 440 of the Fc region of an immunoglobulin. In one embodiment, Fc mutations that can promote antibody hexamerization include one or more mutation(s) in the segment corresponding to E345 to S440 in IgG1. Such one or more mutation(s) can also include mutations corresponding to amino acid residues at amino acid residue positions 345, 430, and/or 440 (e.g., E345, E430 and/or S440 in IgG1). In some embodiments, mutations can include E430G, E430S, E430F, E430T, E345K, E345Q, E345R, E345Y, and S440W. In some embodiments, the mutations include E345K and E430G. These mutations are known as “hexamerization enhancing mutations” in the context of the present invention.

Fc mutations that can reduce effector function include one or more mutation(s) in the amino acid residues L234 and/or L235 to S440 in IgG1. In one embodiment, effector function mutations in the Fc region include L234A and L23SA in IgG1 Fc mutations that can stabilize IgG4 include, but are not limited to, S228, L235 and/or R409 in IgG4. In one embodiment, Fc mutations that can stabilize IgG4 include S228P and L235E or R409K in IgG4. (See also, Vidarsson et al., Front Immunol 2014:5-520 for general discussion of structure and effector functions of IgG subclasses).

In one embodiment, the polypeptide according to the invention is an engineered polypeptide comprising an Fc variant of a wild-type human IgG Fc region, wherein the Fc variant comprises amino acid substitutions at residue positions 228, 234, 235, 345, 409, 430, 440, or a combination thereof, and wherein the amino acid residues are numbered according to the EU index of Kabat. In some embodiments, at least two, three, four, five, six, or seven, amino acid substitutions are made at residue positions 228, 234, 235, 345, 409, 430, 440. In one embodiment, the amino acid at residue position 228 according to the EU index of Kabat is substituted with proline (P) or serine (S). In one embodiment, the amino acid at residue position 234 according to the EU index of Kabat is substituted with alanine (A). In one embodiment, the amino acid at residue position 235 according to the EU index of Kabat is substituted with alanine (A). In one embodiment, glutamate (E) at residue position 345 according to the EU index of Kabat is substituted with lysine (K), glutamine (Q), arginine (R), or tyrosine (Y). In one embodiment, the amino acid at residue position 409 according to the EU index of Kabat is substituted with lysine (K), or arginine (R). In one embodiment, glutamate (E) at residue position 430 according to the EU index of Kabat is substituted with glycine (G), serine (S), phenylalanine (F), or threonine (T). In one embodiment, serine (S) at residue position 440 according to the EU index of Kabat is substituted with tryptophan (W).

In the present specification and claims, the numbering of the residues in an immunoglobulin heavy chain is that of the EU index as in Kabat, et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

Accordingly, the invention provides an antibody variant having a binding region and a full-length or partial Fc domain of an immunoglobulin having one or more hexamerization enhancing mutations and one or more effector function reducing mutations. The antibody variant of the present invention has enhanced receptor clustering and or effector cell signaling compared to an antibody having a wild type Fc domain.

The invention as described herein is further directed to antibodies comprising a variant Fc domain. In one embodiment, the antibody is an anti-GITR antibody comprising a variant Fc domain. Table 1A-1B provides the nucleic acid sequences (SEQ ID NOS: 7-8) and the amino acid sequences (SEQ ID NOS: 9-10), respectively, of the Variable Regions of the Heavy Chain and Light chain of an anti-GITR antibody. In one embodiment, a variant Fc region described herein can be grafted with the Variable Region of an antibody to engineer an antibody of interest, such as an anti-GITR antibody or an anti-CCR4 antibody.

TABLE 1A
Ab #E1-3H7 Variable Region
nucleic acid sequences

V_{H} chain of Ab #E1-3H7 VH
(IGHV3-23*04)
CAGGTGCAGCTGGTGCAGTCTGGGGGAGGC
TTGGTACAGCCTGGGGGGTCCCTGAGACTC
TCCTGTGCAGCCTCTGGATTCACCTTTAGC
AGCCATGCCATGAGCTGGGTCCGCCAGGCT
CCAGGGAAGGGGCTGGAGTGGGTCTCAGCT
ATTAGTGGTAGTGGTGGTAGCACATACTAC
GCAGACTCCGTGAAGGGCCGGTTCACCATC
TCCAGAGACAATTCCAAGAACACGCTGTAT
CTGCAAATGAACAGCCTGAGAGCCGAGGAC
ACGGCCGTATATTACTGTGCGAAAATCGGT
ACGGCGGATGCTTTTGATATCTGGGGCCAA
GGGACCACGGTCACCGTCTCCTCAG
(SEQ ID NO: 7)
V_{L} chain of Ab #E1-3H7 VL
(IGLV1-44*01)
CAGTCTGCCCTGACTCAGCCACCCTCAGTG
TCTGGGACCCCCGGACAGAGGGTCACCATC
TCTTGTTCTGGAGGCGTCCCCAACATCGGA
AGTAATCCTGTAAACTGGTACCTCCACCGC
CCAGGAACGGCCCCCAAACTCCTCATCTAT
AATAGCAATCAGTGGCCCTCAGGGGTCCCT
GACCGATTTTCTGGCTCCAGGTCTGGCACC
TCAGCCTCCCTGGCCATCAGTGGGCTCCAG
TCTGAGGATGAGGCTGATTATTACTGTGCA
GCATGGGATGACAGCCTGGATGGTCTGGTT
TTCGGCGGAGGGACCAAGTTGACCGTCCTA
G
(SEQ ID NO: 8)

TABLE 1B
Ab #E1-3H7 Variable Region
amino acid sequences

V_{H} chain of Ab #E1-3H7
VH (IGHV3-23*04)
QVQLVQSGGGLVQPGGSLRLSCAASGFTFSS
HAMSWVRQAPGKGLEWVSAISGSGGSTYYAD
SVKGRFTISRDNSKNTLYLQMNSLRAEDTAV
YYCAKIGTADAFDIWGQGTTVTVSS
(SEQ ID NO: 9)
V_{L} chain of Ab #E1-3H7
VL (IGLV1-44*01)
QSALTQPPSVSGTPGQRVTISCSGGVPNIGS
NPVNWYLHRPGTAPKLLIYNSNQWPSGVPDR
FSGSRSGTSASLAISGLQSEDEADYYCAAWD
DSLDGLVFGGGTKLTVL
(SEQ ID NO: 10)

Table 1C. below shows the demarcation of the Frameworks and CDRs of the heavy and light chain Variable Region for an anti-GITR antibody based off of SEQ ID NOS: 9-10.

TABLE 1C
anti-GITR E1-3H7 amino
acid sequences

| | | SEQ ID |
|---|---|---|
| | | NO: |
| VH | | |
| FR1 | QVQLVQSGGGLVQPGGSLRLSCAAS | 45 |
| CDR1 | GFTFSSHA | 46 |
| FR2 | MSWVRQAPGKGLEWVSA | 47 |
| CDR2 | ISGSGGST | 48 |
| FR3 | YYADSVKGRFTISRDNSKNTLYL | 49 |
| | QMNSLRAEDTAVYYC | |
| CDR3 | AKIGTADAFDI | 50 |
| FR4 | WGQGTTVTVSS | 51 |
| VL | | |
| FR1 | QSALTQPPSVSGTPGQRVTISCSGG | 52 |
| CDR1 | VPNIGSNP | 53 |
| FR2 | VNWYLHRPGTAPKLLIY | 54 |
| CDR2 | NSN | |
| FR3 | QWPSGVPDRFSGSRSGTSASLAIS | 55 |
| | GLQSEDEADYYC | |
| CDR3 | AAWDDSLDGLV | 56 |
| FR4 | FGGGTKLTVL | 57 |

In one embodiment, the antibody is an anti-CCR4 antibody comprising a variant Fc domain. Table 1D. provides the amino acid sequences (SEQ ID NOS: 11-12) of the Variable Regions of the Heavy Chain and Light chain of an anti-CCR4 antibody. In one embodiment, a variant Fc region described herein can be grafted with the Variable Region of an antibody to engineer an antibody of interest, such as an anti-GITR antibody or an anti-CCR4 antibody.

TABLE 1D
Anti-CCR4 mAb2.3 Variable Region
amino acid sequences
(= affinity maturated, humanized mAb1567)

V_{H} chain of anti-CCR4 mAb2.3
QVQLVQSGAEVKKPGASVKVSCKASGYTFASAWMHWMRQAP
GQGLEWIGWINPGNVNTKYNEKFKGRATLTVDTSTNTAYME
LSSLRSEDTAVYYCARSTYYRPLDYWGQGTLVTVSS
(SEQ ID NO: 11)
V_{L} chain of anti-CCR4 mAb2.3
DIVMTQSPDSLAVSLGERATINCKSSQSILYSSNQKNYLA
WYQQKPGQSPKLLIYWASTRESGVPDRFSGSGSGTDFTLT
ISSLQAEDVAVYYCHQYMSSYTFGQGTKLEIK
(SEQ ID NO: 12)

Table 1E. below shows the demarcation of the Frameworks and CDRs of the heavy and light chain Variable Region for an anti-CCR4 antibody based off of SEQ ID NOS: 11-12.

TABLE 1E
anti-CCR4 mAb2.3amino acid sequences.

| | | SEQ ID NO: |
|---|---|---|
| VH | | |
| FR1 | QVQLVQSGAEVKKPGASVKVSCKAS | 58 |
| CDR1 | GYTFASAW | 59 |
| FR2 | MHWMRQAPGQGLEWIGW | 60 |
| CDR2 | INPGNVNT | 61 |
| FR3 | KYNEKFKGRATLTVDTSTNTAYMELSSLRSEDTAVYYCAR | 62 |
| CDR3 | STYYRPLDY | 63 |
| FR4 | WGQGTLVTVSS | 64 |
| VL | | |
| FR1 | DIVMTQSPDSLAVSLGERATINCKSS | 65 |
| CDR1 | QSILYSSNQKNY | 66 |
| FR2 | LAWYQQKPGQSPKLLIY | 67 |
| CDR2 | WASTRE | 68 |
| FR3 | SGVPDRFSGSGSGTDFTLTISSLQAEDVAVYYC | 69 |
| CDR3 | HQYMSSYT | 70 |
| FR4 | FGQGTKLEIK | 71 |

Table 2A. provides the nucleic acid sequences (SEQ ID NOS: 13-17) for the Constant Region (Fc) of wild type IgG1 heavy chain and light chain. For example, the Fc region described herein can be used to engineer the Fc region of an antibody of interest, such as an anti-GITR antibody or an anti-CCR4 antibody.

TABLE 2A
Ab #E1-3H7 Constant Region nucleic acid sequences-wild
type IgG1 monomer (same for the anti-CCR4 mAb2.3
described herein construct except C_{L})

CH1
ACCAAGGGCCCATCGGTCTTCCCCCTGGCACCCTCCTCCAAGAGCACCTCTGGGGGCAC
AGCGGCCCTGGGCTGCCTGGTCAAGGACTACTTCCCCGAACCGGTGACGGTGTCGTGGA
ACTCAGGCGCCCTGACCAGCGGCGTGCACACCTTCCCGGCTGTCCTACAGTCCTCAGGA
CTCTACTCCCTCAGCAGCGTGGTGACCGTGCCCTCCAGCAGCTTGGGCACCCAGACCTA
CATCTGCAACGTGAATCACAAGCCCAGCAACACCAAGGTGGACAAGAAA (SEQ ID NO:
13)
Hinge
GCAGAGCCCAAATCTTGTGACAAAACTCACACATGCCCACCGTGCCCA (SEQ ID NO: 14)
CH2
GCACCTGAACTCCTGGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAG
ACCCTGAGGTCAAGTTCAACTGGTACGTGGACGGCGTGGAGGTGCATAATGCCAAGAC
AAAGCCGCGGGAGGAGCAGTACAACAGCACGTACCGTGTGGTCAGCGTCCTCACCGTC
CTGCACCAGGACTGGCTGAATGGCAAGGAGTACAAGTGCAAGGTCTCCAACAAAGCCC
TCCCAGCCCCCATCGAGAAAACCATCTCCAAAGCCAAA (SEQ ID NO: 15)
CH3
GGGCAGCCCCGAGAACCACAGGTGTACACCCTGCCCCCATCCCGGGATGAGCTGACCA
AGAACCAGGTCAGCCTGACCTGCCTGGTCAAAGGCTTCTATCCCAGCGACATCGCCGTG
GAGTGGGAGAGCAATGGGCAGCCGGAGAACAACTACAAGACCACGCCTCCCGTGCTGG
ACTCCGACGGCTCCTTCTTCCTCTACAGCAAGCTCACCGTGGACAAGAGCAGGTGGCAG
CAGGGGAACGTCTTCTCATGCTCCGTGATGCATGAGGCTCTGCACAACCACTACACGCA
GAAGAGCCTCTCCCTGTCTCCGGGTAAATGA (SEQ ID NO: 16)
C_{L}
GGTCAGCCCAAGGCTGCCCCCTCGGTCACTCTGTTCCCGCCCTCCTCTGAGGAGCTTCA
AGCCAACAAGGCCACACTGGTGTGTCTCATAAGTGACTTCTACCCGGGAGCCGTGACA
GTGGCCTGGAAGGCAGATGGCAGCCCCGTCAAGGCGGGAGTGGAGACCACCACACCCT
CCAAACAAAGCAACAACAAGTACGCGGCCAGCAGCTATCTGAGCCTGACGCCTGAGCA
GTGGAAGTCCCACAGAAGCTACAGCTGCCAGGTCACGCATGAAGGGAGCACCGTGGAG
AAGACAGTGGCCCCTACAGAATGTTCATGA (SEQ ID NO: 17)

In one embodiment, the Fc region of the light chain described herein can be used to engineer the Fc region of an antibody of interest, such as an anti-GITR antibody or an anti-CCR4 antibody. In one embodiment, the Fc region of the light chain (C_{L (kappa)}) comprises the nucleic acid sequence of SEQ ID NO: 43:

CGTACGGTGGCTGCACCATCTGTCTTCATCTTCCCGCCATCTGATGAGC
AGTTGAAATCTGGAACTGCCTCTGTTGTGTGCCTGCTGAATAACTTCTA
TCCCAGAGAGGCCAAAGTACAGTGGAAGGTGGATAACGCCCTCCAATCG
GGTAACTCCCAGGAGAGTGTCACAGAGCAGGACAGCAAGGACAGCACCT
ACAGCCTCAGCAGCACCCTGACGCTGAGCAAAGCAGACTACGAGAAACA
CAAAGTCTACGCCTGCGAAGTCACCCATCAGGGCCTGAGCTCGCCCGTC
ACAAAGAGCTTCAACAGGGGAGAGTGTTGA

In one embodiment, the Fc region of the light chain (C_{L (kappa)}) comprises the amino acid sequence of SEQ ID NO: 44:

RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC

Table 2B. provides the amino acid sequences (SEQ ID NOS: 18-22) for the Constant Region (Fc) of wild type IgG1 heavy chain and light chain. For example, the Fc region described herein can be used to engineer the Fc region of an antibody of interest, such as an anti-GITR antibody or an anti-CCR4 antibody.

TABLE 2B
Ab #E1-3H7 Constant Region amino acid sequences-wild
type IgG1 monomer (same for the anti-CCR4 mAb2.3 construct
except C_{L}). The bolded residues in CH2 and CH3, for example,
are wild type residues that can be mutated to make different
IgG1 mutants (yellow highlighted residues in Tables 3-5).

CH1
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKK (SEQ ID NO: 18)
Hinge
AEPKSCDKTHTCPPCP (SEQ ID NO: 19)
CH2
APELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK (SEQ ID NO: 20)
CH3
GQPREPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK (SEQ ID NO: 21)
C_{L}
GQPKAAPSVTLFPPSSEELQANKATLVCLISDFYPGAVTVAWKADGSPVKAGVETTTPSKQ
SNNKYAASSYLSLTPEQWKSHRSYSCQVTHEGSTVEKTVAPTECS (SEQ ID NO: 22)

Table 3A. provides the nucleic acid sequences (SEQ ID NO: 23) for a variant Constant Region (Fc) of IgG1 heavy chain and light chain. The yellow-highlighted residues in indicate mutations introduced into the Fc region to make an IgG1 Fc variant. For example, the Fc region described herein can be used to engineer a variant Fc region of an antibody of interest, such as an anti-GITR antibody or an anti-CCR4 antibody.

TABLE 3A
Ab #E1-3H7 Constant Region nucleic acid sequences-IgG1
LALA mutant monomer (same for the anti-CCR4 mAb2.3
construct except C_{L})

CH1
Same as wild type (see Table 2A)
Hinge
Same as wild type (see Table 2A)
CH2
GCACCTGAAGCCGCCGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAG
ACCCTGAGGTCAAGTTCAACTGGTACGTGGACGGCGTGGAGGTGCATAATGCCAAGAC
AAAGCCGCGGGAGGAGCAGTACAACAGCACGTACCGTGTGGTCAGCGTCCTCACCGTC
CTGCACCAGGACTGGCTGAATGGCAAGGAGTACAAGTGCAAGGTCTCCAACAAAGCCC
TCCCAGCCCCCATCGAGAAAACCATCTCCAAAGCCAAA (SEQ ID NO: 23)
CH3
Same as wild type (see Table 2A)
C_{L}
Same as wild type (see Table 2A)

Table 3B. provides the amino acid sequences (SEQ ID NO: 24) for a variant Constant Region (Fc) of IgG1 heavy chain and light chain. The yellow-highlighted residues in indicate mutations introduced into the Fc region to make an IgG1 Fc variant. For example, the Fc region described herein can be used to engineer a variant Fc region of an antibody of interest, such as an anti-GITR antibody or an anti-CCR4 antibody.

TABLE 3B
Ab #E1-3H7 Constant Region amino acid sequences-
IgG1 LALA mutant monomer (same for the anti-CCR4
mAb2.3 construct except C_{L})

CH1
Same as wild type (see Table 2B)
Hinge
Same as wild type (see Table 2B)
CH2
APEAAGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAK (SEQ ID NO: 24)
CH3
Same as wild type (see Table 2B)
C_{L}
Same as wild type (see Table 2B)

Table 4A. provides the nucleic acid sequences (SEQ ID NO: 25) for a variant Constant Region (Fc) of IgG1 heavy chain and light chain. The yellow-highlighted residues in indicate mutations introduced into the Fc region to make an IgG1 Fc variant. For example, the Fc region described herein can be used to engineer a variant Fc region of an antibody of interest, such as an anti-GITR antibody or an anti-CCR4 antibody.

TABLE 4A
Ab #E1-3H7 Constant Region nucleic acid sequences-
IgG1 WT hexamer (same for the anti-CCR4 mAb2.3
construct except C_{L})

CH1
Same as wild type (see Table 2A)
Hinge
Same as wild type (see Table 2A)
CH2
Same as wild type (see Table 2A)
CH3
GGGCAGCCCCGAAAGCCACAGGTGTACACCCTGCCCCCATCCCGGGATGAGCTGACCA
AGAACCAGGTCAGCCTGACCTGCCTGGTCAAAGGCTTCTATCCCAGCGACATCGCCGTG
GAGTGGGAGAGCAATGGGCAGCCGGAGAACAACTACAAGACCACGCCTCCCGTGCTGG
ACTCCGACGGCTCCTTCTTCCTCTACAGCAAGCTCACCGTGGACAAGAGCAGGTGGCAG
CAGGGGAACGTCTTCTCATGCTCCGTGATGCATGGAGCTCTGCACAACCACTACACGCA
GAAGAGCCTCTCCCTGTCTCCGGGTAAATGA (SEQ ID NO: 25)
C_{L}
Same as wild type (see Table 2A)

Table 4B. provides the amino acid sequences (SEQ ID NO: 26) for a variant Constant Region (Fc) of IgG1 heavy chain and light chain. The yellow-highlighted residues in indicate mutations introduced into the Fc region to make an IgG1 Fc variant. For example, the Fc region described herein can be used to engineer a variant Fc region of an antibody of interest, such as an anti-GITR antibody or an anti-CCR4 antibody.

TABLE 4B
Ab #E1-3H7 Constant Region amino acid sequences-
IgG1 WT hexamer (same for the anti-CCR4 mAb2.3
construct except C_{L})

CH1
Same as wild type (see Table 2B)
Hinge
Same as wild type (see Table 2B)
CH2
Same as wild type (see Table 2B)
CH3
GQPRKPQVYTLPPSRDELTKNQVSLTCLVKGFYPSDIAVEWESNGQPENN
YKTTPPVLDSDGSFFLYSKLTVDKSRWQQGNVFSCSVMHGALHNHYTQKS
LSLSPGK (SEQ ID NO: 26)
C_{L}
Same as wild type (see Table 2B)

Table 5A. provides the nucleic acid sequences (SEQ ID NOS: 27-28) for a variant Constant Region (Fc) of IgG1 heavy chain and light chain. The yellow-highlighted residues in indicate mutations introduced into the Fc region to make an IgG1 Fc variant. For example, the Fc region described herein can be used to engineer a variant Fc region of an antibody of interest, such as an anti-GITR antibody or an anti-CCR4 antibody.

TABLE 5A
Ab #E1-3H7 Constant Region nucleic acid sequences-IgG1
LALA hexamer (same for the anti-CCR4 mAb2.3 construct
except C_{L})

CH1
Same as wild type (see Table 2A)
Hinge
Same as wild type (see Table 2A)
CH2 (identical to CH2 in Table 3A)
GCACCTGAAGCCGCCGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAG
ACCCTGAGGTCAAGTTCAACTGGTACGTGGACGGCGTGGAGGTGCATAATGCCAAGAC
AAAGCCGCGGGAGGAGCAGTACAACAGCACGTACCGTGTGGTCAGCGTCCTCACCGTC
CTGCACCAGGACTGGCTGAATGGCAAGGAGTACAAGTGCAAGGTCTCCAACAAAGCCC
TCCCAGCCCCCATCGAGAAAACCATCTCCAAAGCCAAA (SEQ ID NO: 27)
CH3 (identical to CH3 in Table 4A)
GGGCAGCCCCGAAAGCCACAGGTGTACACCCTGCCCCCATCCCGGGATGAGCTGACCA
AGAACCAGGTCAGCCTGACCTGCCTGGTCAAAGGCTTCTATCCCAGCGACATCGCCGTG
GAGTGGGAGAGCAATGGGCAGCCGGAGAACAACTACAAGACCACGCCTCCCGTGCTGG
ACTCCGACGGCTCCTTCTTCCTCTACAGCAAGCTCACCGTGGACAAGAGCAGGTGGCAG
CAGGGGAACGTCTTCTCATGCTCCGTGATGCATGGAGCTCTGCACAACCACTACACGCA
GAAGAGCCTCTCCCTGTCTCCGGGTAAATGA (SEQ ID NO: 28)
C_{L}
Same as wild type (see Table 2A)

Table 5B. provides the amino acid sequences (SEQ ID NOS: 29-30) for a variant Constant Region (Fc) of IgG1 heavy chain and light chain. The yellow-highlighted residues in indicate mutations introduced into the Fc region to make an IgG1 Fc variant. For example, the Fc region described herein can be used to engineer a variant Fc region of an antibody of interest, such as an anti-GITR antibody or an anti-CCR4 antibody.

TABLE 5B
Ab #E1-3H7 Constant Region amino acid sequences-
IgG1 LALA hexamer (same for the anti-CCR4 mAb2.3
construct except C_{L})

CH1
Same as wild type (see Table 2B)
Hinge
Same as wild type (see Table 2B)
CH2 (identical to CH2 in Table 3B)
APEAAGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK (SEQ ID NO: 29)
CH3 (identical to CH3 in Table 4B)
GQPRKPQVYTLPPSRDELTKNQVSLTCLVKGFYPSDIAVEWESNGQPENNYKTTPPVLDSD
GSFFLYSKLTVDKSRWQQGNVFSCSVMHGALHNHYTQKSLSLSPGK (SEQ ID NO: 30)
C_{L}
Same as wild type (see Table 2B)

Table 6A. provides the nucleic acid sequences (SEQ ID NOS: 31-35) for the Constant Region (Fc) of stabilized IgG4 heavy chain and light chain. Yellow highlighted residues are mutations that were introduced to stabilize IgG4. For example, the Fc region described herein can be used to engineer the Fc region of an antibody of interest, such as an anti-GITR antibody or an anti-CCR4 antibody.

TABLE 6A
Ab #E1-3H7 Constant Region nucleic acid sequences-
sIgG4 monomer (same for the anti-CCR4 mAb2.3
construct except C_{L})

CH1
GCTAGCACCAAGGGCCCATCCGTCTTCCCCCTGGCGCCCTGCTCCAGGAGCACCTCCGA
GAGCACAGCCGCCCTGGGCTGCCTGGTCAAGGACTACTTCCCCGAACCGGTGACGGTGT
CGTGGAACTCAGGCGCCCTGACCAGCGGCGTGCACACCTTCCCGGCTGTCCTACAGTCC
TCAGGACTCTACTCCCTCAGCAGCGTGGTGACCGTGCCCTCCAGCAGCTTGGGCACGAA
GACCTACACCTGCAACGTAGATCACAAGCCCAGCAACACCAAGGTGGACAAGAGAGTT
(SEQ ID NO: 31)
Hinge
GAGTCCAAATATGGTCCCCCATGCCCACCATGCCCA (SEQ ID NO: 32)
CH2
GCACCTGAGTTCCTGGGGGGACCATCAGTCTTCCTGTTCCCCCCAAAACCCAAGGACAC
TCTCATGATCTCCCGGACCCCTGAGGTCACGTGCGTGGTGGTGGACGTGAGCCAGGAAG
ACCCCGAGGTCCAGTTCAACTGGTACGTGGATGGCGTGGAGGTGCATAATGCCAAGAC
AAAGCCGCGGGAGGAGCAGTTCAACAGCACGTACCGTGTGGTCAGCGTCCTCACCGTC
CTGCACCAGGACTGGCTGAACGGCAAGGAGTACAAGTGCAAGGTCTCCAACAAAGGCC
TCCCGTCCTCCATCGAGAAAACCATCTCCAAAGCCAAA (SEQ ID NO: 33)
CH3
GGGCAGCCCCGAGAGCCACAGGTGTACACCCTGCCCCCATCCCCGGAGGAGATGACCA
AGAACCAGGTCAGCCTGACCTGCCTGGTCAAAGGCTTCTACCCCAGCGACATCGCCGTG
GAGTGGGAGAGCAATGGGCAGCCGGAGAACAACTACAAGACCACGCCTCCCGTGCTGG
ACTCCGACGGCTCCTTCTTCCTCTACAGCAAGCTAACCGTGGACAAGAGCAGGTGGCAG
GAGGGGAATGTCTTCTCATGCTCCGTGATGCATGAGGCTCTGCACAACCACTACACACA
GAAGAGCCTCTCCCTGTCTCTGGGTAAATGA (SEQ ID NO: 34)
C_{L}
GGTCAGCCCAAGGCTGCCCCCTCGGTCACTCTGTTCCCGCCCTCCTCTGAGGAGCTTCA
AGCCAACAAGGCCACACTGGTGTGTCTCATAAGTGACTTCTACCCGGGAGCCGTGACA
GTGGCCTGGAAGGCAGATGGCAGCCCCGTCAAGGCGGGAGTGGAGACCACCACACCCT
CCAAACAAAGCAACAACAAGTACGCGGCCAGCAGCTATCTGAGCCTGACGCCTGAGCA
GTGGAAGTCCCACAGAAGCTACAGCTGCCAGGTCACGCATGAAGGGAGCACCGTGGAG
AAGACAGTGGCCCCTACAGAATGTTCATGA ((SEQ ID NO: 35)

Table 6B. provides the amino acid sequences (SEQ ID NOS: 36-40) for the Constant Region (Fc) of stabilized IgG4 heavy chain and light chain. Yellow highlighted residues are mutations that were introduced to stabilize IgG4. The bolded residues are wild type residues that can be mutated to make an sIgG4 hexamer in Table 7. For example, the Fc region described herein can be used to engineer the Fc region of an antibody of interest, such as an anti-GITR antibody or an anti-CCR4 antibody.

TABLE 6B
Ab #E1-3H7 Constant Region amino acid sequences-sIgG4 monomer
(same for the anti-CCR4 mAb2.3 construct except C_{L})

CH1
ASTKGPSVFPLAPCSRSTSESTAALGCLVKDYFPEPVTVSWNSGALTSGVHTFPAVLQSSGL
YSLSSVVTVPSSSLGTKTYTCNVDHKPSNTKVDKRV (SEQ ID NO: 36)
Hinge
ESKYGPPCPPCP (SEQ ID NO: 37)
CH2
APEFLGGPSVFLFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAK
(SEQ ID NO: 38)
CH3
GQPREPQVYTLPPSPEEMTKNQVSLTCLVKGFYPSDIAVEWESNGQPENNYKTTPPVLDSD
GSFFLYSKLTVDKSRWQEGNVFSCSVMHEALHNHYTQKSLSLSLGK (SEQ ID NO: 39)
C_{L}
GQPKAAPSVTLFPPSSEELQANKATLVCLISDFYPGAVTVAWKADGSPVKAGVETTTPSKQ
SNNKYAASSYLSLTPEQWKSHRSYSCQVTHEGSTVEKTVAPTECS (SEQ ID NO: 40)

Table 7A. provides the nucleic acid sequences (SEQ ID NOS: 31-33, 35, and 41) for a variant Constant Region (Fc) of stabilized IgG4 heavy chain and light chain. Yellow highlighted residues are mutations that were introduced to stabilize IgG4. The bolded residues are wild type residues that can be mutated to make an sIgG4 hexamer in Table 7. For example, the Fc region described herein can be used to engineer the Fc region of an antibody of interest, such as an anti-GITR antibody or an anti-CCR4 antibody.

TABLE 7A
Ab #E1-3H7 Constant Region nucleic acid sequences-sIgG4
hexamer (same for the anti-CCR4 mAb2.3 construct except C_{L})

CH1 (same as SEQ ID NO: 31 in Table 6A)
GCTAGCACCAAGGGCCCATCCGTCTTCCCCCTGGCGCCCTGCTCCAGGAGCACCTCCGA
GAGCACAGCCGCCCTGGGCTGCCTGGTCAAGGACTACTTCCCCGAACCGGTGACGGTGT
CGTGGAACTCAGGCGCCCTGACCAGCGGCGTGCACACCTTCCCGGCTGTCCTACAGTCC
TCAGGACTCTACTCCCTCAGCAGCGTGGTGACCGTGCCCTCCAGCAGCTTGGGCACGAA
GACCTACACCTGCAACGTAGATCACAAGCCCAGCAACACCAAGGTGGACAAGAGAGTT
Hinge (same as SEQ ID NO: 32 in Table 6A)
GAGTCCAAATATGGTCCCCCATGCCCACCATGCCCA
CH2 (same as SEQ ID NO: 33 in Table 6A)
GCACCTGAGTTCCTGGGGGGACCATCAGTCTTCCTGTTCCCCCCAAAACCCAAGGACAC
TCTCATGATCTCCCGGACCCCTGAGGTCACGTGCGTGGTGGTGGACGTGAGCCAGGAAG
ACCCCGAGGTCCAGTTCAACTGGTACGTGGATGGCGTGGAGGTGCATAATGCCAAGAC
AAAGCCGCGGGAGGAGCAGTTCAACAGCACGTACCGTGTGGTCAGCGTCCTCACCGTC
CTGCACCAGGACTGGCTGAACGGCAAGGAGTACAAGTGCAAGGTCTCCAACAAAGGCC
TCCCGTCCTCCATCGAGAAAACCATCTCCAAAGCCAAA
CH3
GGGCAGCCCCGAAAGCCACAGGTGTACACCCTGCCCCCATCCCCGGAGGAGATGACCA
AGAACCAGGTCAGCCTGACCTGCCTGGTCAAAGGCTTCTACCCCAGCGACATCGCCGTG
GAGTGGGAGAGCAATGGGCAGCCGGAGAACAACTACAAGACCACGCCTCCCGTGCTGG
ACTCCGACGGCTCCTTCTTCCTCTACAGCAAGCTAACCGTGGACAAGAGCAGGTGGCAG
GAGGGGAATGTCTTCTCATGCTCCGTGATGCATGGAGCTCTGCACAACCACTACACACA
GAAGAGCCTCTCCCTGTCTCTGGGTAAATGA
(SEQ ID NO 41)
C_{L} (same as SEQ ID NO: 35 in Table 6A)
GGTCAGCCCAAGGCTGCCCCCTCGGTCACTCTGTTCCCGCCCTCCTCTGAGGAGCTTCA
AGCCAACAAGGCCACACTGGTGTGTCTCATAAGTGACTTCTACCCGGGAGCCGTGACA
GTGGCCTGGAAGGCAGATGGCAGCCCCGTCAAGGCGGGAGTGGAGACCACCACACCCT
CCAAACAAAGCAACAACAAGTACGCGGCCAGCAGCTATCTGAGCCTGACGCCTGAGCA
GTGGAAGTCCCACAGAAGCTACAGCTGCCAGGTCACGCATGAAGGGAGCACCGTGGAG
AAGACAGTGGCCCCTACAGAATGTTCATGA

Table 7B. provides the amino acid sequences (SEQ ID NOS: 36-40) for the Constant Region (Fc) of stabilized IgG4 heavy chain and light chain. Yellow highlighted residues are mutations that were introduced to stabilize IgG4. The bolded residues are wild type residues that can be mutated to make an sIgG4 hexamer in Table 7. For example, the Fc region described herein can be used to engineer the Fc region of an antibody of interest, such as an anti-GITR antibody or an anti-CCR4 antibody.

TABLE 7B
Ab #E1-3H7 Constant Region amino acid sequences-sIgG4
hexamer (same for the anti-CCR4 mAb2.3 construct except C_{L})

CH1 (same as SEQ ID NO: 36 in Table 6B)
ASTKGPSVFPLAPCSRSTSESTAALGCLVKDYFPEPVTVSWNSGALTSGVHTFPAVLQSSGL
YSLSSVVTVPSSSLGTKTYTCNVDHKPSNTKVDKRV
Hinge (same as SEQ ID NO: 37 in Table 6B)
ESKYGPPCPPCP
CH2 (same as SEQ ID NO: 38 in Table 6B)
APEFLGGPSVFLFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAK
CH3
GQPRKPQVYTLPPSPEEMTKNQVSLTCLVKGFYPSDIAVEWESNGQPENNYKTTPPVLDSD
GSFFLYSKLTVDKSRWQEGNVFSCSVMHGALHNHYTQKSLSLSLGK (SEQ ID NO: 42)
C_{L} (same as SEQ ID NO: 40 in Table 6B)
GQPKAAPSVTLFPPSSEELQANKATLVCLISDFYPGAVTVAWKADGSPVKAGVETTTPSKQ
SNNKYAASSYLSLTPEQWKSHRSYSCQVTHEGSTVEKTVAPTECS

Antibody variants having one or more hexamerization enhancing mutations and one or more effector function reducing will have improved therapeutic potential. In particular antibodies that act as agonists or antagonists after binding to the target cell surface will have increase biological activity. This is especially true when cell surface receptor clustering is required for their biological function. The enhanced receptor clustering and or effector cell signaling or the antibody variants of the invention translates to practical clinical benefits, for example, lowering the effective doses of human monoclonal antibodies to achieve therapeutic effects as well as using antibodies with lower affinity antibodies.

Accordingly, the invention also provides methods of using the antibody variants of the invention in therapeutic methods to treat cancer, autoimmune disorders, inflammatory disorders, neurologie disease. cardiovascular disease, infectious diseases and to direct stem cell linage pathways. The term “treating” can refer to partially or completely alleviating, ameliorating, improving, relieving, delaying onset of, inhibiting progression of, reducing severity of, and/or reducing incidence of one or more symptoms, features, or clinical manifestations of a particular disease, disorder, and/or condition. Treatment can be administered to a subject who does not exhibit signs of a disease, disorder, and/or condition (e.g., prior to an identifiable disease, disorder, and/or condition), and/or to a subject who exhibits only early signs of a disease, disorder, and/or condition for the purpose of decreasing the risk of developing pathology associated with the disease, disorder, and/or condition. In some embodiments, treatment comprises enhancing cellular signaling or inducing receptor clustering of a cell.

The antibody variants of the invention can be specific for any target of interest. For example the target of interest can be a tumor-associated surface antigen, such as ErbB2 (HER2/neu), carcinoembryonic antigen (CEA), epithelial cell adhesion molecule (EpCAM), epidermal growth factor receptor (EGFR), EGFR variant III (EGFRvIII), CD19, CD20, CD30, CD40, disialoganglioside GD2, ductal-epithelial mucine, gp36, TAG-72, glycosphingolipids, glioma-associated antigen, .beta.-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate specific antigen (PSA), PAP, NY-ESO-1, LAGA-1a, p53, prostein (P501s), PSMA, surviving and telomerase, prostate-carcinoma tumor antigen-1 (PCTA-1), MAGE, ELF2M, neutrophil elastase, ephrin B2, CD22, insulin growth factor (IGF1)-I, IGF-II, IGFI receptor, mesothelia, a major histocompatibility complex (MHC) molecule presenting a tumor-specific peptide epitope, 5T4, ROR1, Nkp30, NKG2D, tumor stromal antigens, the extra domain A (EDA) and extra domain B (EDB) of fibronectin and the Al domain of tenascin-C (TnC A1) and fibroblast associated protein (fap); a lineage-specific or tissue specific antigen such as CD3, CD4, CD8, CD24, CD25, CD28,CD33, CD34, CD133. CD138, CTLA-4, B7-1 (CD80), B7-2 (CD86), endoglin, a major histocompatibility complex (MHC) molecule, BCMA (CD269, TNFRSF 17), or a virus-specific surface antigen such as an HIV-specific antigen (such as HIV gp120); an EBV-specific antigen, a CMV-specific antigen, a HPV-specific antigen, a Lasse Virus-specific antigen, an Influenza Virus-specific antigen as well as any derivate or variant of these surface markers.

Preferably, the antibody is specific for BCMA, CAIX, CCR4, PD-L1, PD-L2, PD1, Glucocorticoid-Induced Tumor Necrosis Factor Receptors (GITR), TIGIT, Severe acute respiratory syndrome (SARS), influenza, flavivirus or Middle East Respiratory Syndrome (MERS).

Exemplary antibodies useful in constructing the antibody variants according to the invention includes antibodies disclosed in for example: WO/2005/060520, WO/2006/089141, WO/2007/065027, WO/2009/086514, WO/2009/079259, WO/2011/153380, WO/2014/055897, WO 2015/143194, WO 2015/164865, WO 2013/166500, and WO 2014/144061; PCT/US2015/054202, PCT/US2015/054010 and 62/144,729 the contents of which are hereby incorporated by reference in their entireties.

Antibodies of the invention and fragments thereof can be synthesized, engineered,

and/or produced using nucleic acids, such as those described in the tables herein. In one embodiment, the nucleic acid has a sequence comprising nucleotides disclosed in Tables 1A, 2A, 3A, 4A, 5A, 6A, 7A, SEQ ID NO: 43, or a combination thereof. In another embodiment, the nucleic acid has a sequence at least 60%, at least 65%, at least 70%, at least 80%, at least 85%, at least 90%, at least 91%, at least 92%, at least 93%, at least 94%, at least 95%, at least 96%, at least 97%, at least 98%, at least 99%, or 100% identical to a nucleic acid sequence disclosed in Tables 1A, 2A, 3A, 4A, 5A, 6A, 7A, SEQ ID NO: 43, or a combination thereof. It will be appreciated that the invention includes portions and variants of the sequences specifically disclosed herein. For example, forms of codon optimized sequences can be used in embodiments.

The encoding sequence can be present, for example, in a replicating or non-replicating adenoviral vector, an adeno-associated virus vector, an attenuated mycobacterium tuberculosis vector, a Bacillus Calmette Guerin (BCG) vector, a vaccinia or Modified Vaccinia Ankara (MVA) vector, another pox virus vector, recombinant polio and other enteric virus vector, Salmonella species bacterial vector, Shigella species bacterial vector, Venezuclean Equine Encephalitis Virus (VEE) vector, a Semliki Forest Virus vector, or a Tobacco Mosaic Virus vector. The encoding sequence, can also be expressed as a DNA plasmid with, for example, an active promoter such as a CMV promoter. Other live vectors can also be used to express the sequences of the invention. Expression of the antibody of the invention can be induced in a subject's own cells, by introduction into those cells of nucleic acids that encode the antibody, preferably using codons and promoters that optimize expression in human cells.

Embodiments of the invention include cells that express the antibody variants of the invention (i.e, CARTs). The cell may be of any kind, including an immune cell capable of expressing the antibody variants for cancer therapy or a cell, such as a bacterial cell, that harbors an expression vector that encodes the CAR. As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a eukaryotic cell that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells can refer to a cell into which an exogenous nucleic acid sequence, such as, for example, a vector, has been introduced. Therefore, recombinant cells are distinguishable from naturally occurring cells which do not contain a recombinantly introduced nucleic acid. In embodiments of the invention, a host cell is a T cell, including a cytotoxic T cell (also known as TC, Cytotoxic T Lymphocyte, CTL, T-Killer cell, cytolytic T cell, CD8+ T-cells or killer T cell); CD4+ T cells, NK cells and NKT cells are also encompassed in the invention.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

The cells can be autologous cells, syngeneic cells, allogenic cells and even in some cases, xenogeneic cells.

In many situations one may wish to be able to kill the modified CTLs, where one wishes to terminate the treatment, the cells become neoplastic, in research where the absence of the cells after their presence is of interest, or other event. For this purpose one can provide for the expression of certain gene products in which one can kill the modified cells under controlled conditions, such as inducible suicide genes.

The invention further includes CARTs that are modified to secrete one or more polypeptides. The polypeptide can be for example an antibody or cytokine. Preferably, the antibody is specific for CAIX, GITR, PD-L1, PD-L2, PD-1, CCR4 or TIGIT.

Armed CARTs have the advantage of simultaneously secreting a polypeptide at the targeted site, e.g. tumor site.

Armed CART can be constructed by including a nucleic acid encoding the polypeptide of interest after the intracellular signaling domain. Preferably, there is an internal ribosome entry site, (IRES), positioned between the intracellular signaling domain and the polypeptide of interest. One skilled in the art can appreciate that more than one polypeptide can be expressed by employing multiple IRES sequences in tandem.

The antibodies comprising the engineered polypeptides may be purified, such as from cells or from recombinant systems, using a variety of well-known techniques for isolating and purifying proteins. See, for example, antibody purification methods in Zola, Monoclonal Antibodies: Preparation and Use of Monoclonal Antibodies and Engineered Antibody Derivatives (Basics: From Background to Bench), Springer-Verlag Ltd., New York, 2000; Basic Methods in Antibody Production and Characterization, Chapter 11, “Antibody Purification Methods,” Howard and Bethell, Eds., CRC Press, 2000; Antibody Engineering (Springer Lab Manual), Kontermann and Dubel, Eds., Springer-Verlag, 2001; each of which are incorporated by reference herein in their entireties.

The antibodies, fragments, and antibody derivatives, for example chimeric antibodies or humanized antibodies, described herein can be formulated as a composition (e.g., a pharmaceutical composition), such as those for use in a subject. Suitable compositions can comprise the antibody or fragment (or derivative thereof) dissolved or dispersed in a pharmaceutically acceptable carrier (e.g., an aqueous medium).

A pharmaceutically acceptable carrier can comprise any and all solvents, dispersion media, coatings, isotonic and absorption delaying agents, and the like, compatible with pharmaceutical administration. The use of such media and agents for pharmaceutically active substances is well known in the art. Any conventional media or agent that is compatible with the antibody can be used. Supplementary active agents can also be incorporated into the compositions. Non-limiting examples of pharmaceutically acceptable carriers comprise solid or liquid fillers, diluents, and encapsulating substances, including but not limited to lactose, dextrose, sucrose, sorbitol, mannitol, xylitol, erythritol, maltitol, starches, gum acacia, alginate, gelatin, calcium phosphate, calcium silicate, cellulose, methyl cellulose, microcrystalline cellulose, polyvinylpyrrolidone, water, methyl benzoate, propyl benzoate, talc, magnesium stearate, and mineral oil.

A pharmaceutical composition of the invention can be sterile, and can be formulated to be compatible with its intended route of administration. Examples of routes of administration include parenteral, e.g., intravenous, intradermal, subcutaneous, oral (e.g., inhalation), transdermal (topical), transmucosal, and rectal administration.

For example, pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the antibody in the required amount in an appropriate solvent with one or a combination of ingredients enumerated herein, as required, followed by filtered sterilization. Generally, dispersions are prepared by incorporating the antibody into a sterile vehicle which contains a basic dispersion medium and the required other ingredients from those enumerated herein.

As another example, oral compositions generally include an inert diluent or an edible carrier. They can be enclosed in gelatin capsules or compressed into tablets. For the purpose of oral therapeutic administration, the antibody can be incorporated with excipients and used in the form of tablets, troches, or capsules.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The antibodies or fragments (or derivatives thereof) can also be formulated as a composition appropriate for topical administration to the skin or mucosa (e.g., intrarectal or intravaginal administration). Such compositions can take the form of liquids, ointments, creams, gels and pastes. The antibodies or fragments (or derivatives thereof) can also be formulated as a composition appropriate for intranasal administration. Standard formulation techniques can be used in preparing suitable compositions.

Antibodies and/or compositions of the invention can be administered to the subject one time (e.g., as a single injection or deposition). Alternatively, administration can be once or twice daily to a subject in need thereof for a period of from about 2 to about 28 days, or from about 7 to about 10 days, or from about 7 to about 15 days. It can also be administered once or twice daily to a subject for a period of 1, 2, 3, 4, 5, 6, 7, 8, 9, 10, 11, 12 times per year, or a combination thereof.

Therapeutically effective dose ranges can depend on the antibody or fragment (or derivative thereof and on the nature of the formulation and route of administration.

Optimum doses can be determined by one skilled in the art without undue experimentation, and can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion. For example, therapeutically effective doses of antibodies in the range of about 0.1-1000 mg/kg body weight can be used. Preferably, doses of antibodies in the range of about 1-50 mg/kg can be used.

An antibody or nucleic acid of the present invention can also be provided in a kit. In one embodiment, the kit includes (a) a container that contains a composition that includes the antibody, and optionally (b) informational material. The informational material can be descriptive, instructional, marketing or other material that relates to the methods described herein and/or the use of the agents for therapeutic benefit. In an embodiment, the kit includes also includes a second agent for treating a subject afflicted with a disease or condition. For example, the kit includes a first container that contains a composition that includes the polypeptide, and a second container that includes the second agent.

The informational material of the kits is not limited in its form. In one embodiment, the informational material can include information about production of the antibody, molecular weight of the antibody, concentration, date of expiration, batch or production site information, and so forth. In one embodiment, the informational material relates to methods of administering the polypeptide or nucleic acid encoding the same, e.g., in a suitable dose, dosage form, or mode of administration (e.g., a dose, dosage form, or mode of administration described herein), to treat a subject. The information can be provided in a variety of formats, include printed text, computer readable material, video recording, or audio recording, or information that provides a link or address to substantive material.

In addition to the antibody or nucleic acid encoding the same, the composition in the kit can include other ingredients, such as a solvent or buffer, a stabilizer, or a preservative. The antibody or nucleic acid can be provided in any form, e.g., liquid, dried or lyophilized form, preferably substantially pure and/or sterile. When provided in a liquid solution, the liquid solution preferably is an aqueous solution. When provided as a dried form, reconstitution generally is by the addition of a suitable solvent. The solvent, e.g., sterile water or buffer, can optionally be provided in the kit.

The kit can include one or more containers for the antibody, nucleic acid, or compositions comprising the same. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers. each containing one or more unit dosage forms (e.g., a dosage form described herein) of the antibodies or nucleic acids. The containers can include a combination unit dosage, e.g., a unit that includes both the antibody and the second agent. e.g., in a desired ratio. For example, the kit includes a plurality of syringes, ampules, foil packets, blister packs, or medical devices, e.g., each containing a single combination unit dose. The containers of the kits can be air tight, waterproof (e.g., impermeable to changes in moisture or evaporation), and/or light-tight. The kit optionally includes a device suitable for administration of the composition, e.g., a syringe or other suitable delivery device. The device can be provided pre-loaded or can be empty, but suitable for loading.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

In the present specification and claims, the numbering of the residues in an immunoglobulin heavy chain is that of the EU index as in Kabat, et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

“Affinity” can refer to, for example, the strength of the sum total of noncovalent interactions between a single binding site of a molecule (e.g., an antibody) and its binding partner (e.g., an antigen or an Fc receptor). Unless indicated otherwise, “binding affinity” can refer to intrinsic binding affinity which reflects a 1:1 interaction between members of a binding pair (e.g., antibody/Fc receptor or antibody and antigen). The affinity of a molecule X for its partner Y can be represented by the dissociation constant (Kd). Affinity can be measured by common methods known in the art, including those described herein. Further. see Yang, Danlin, et al. “Determination of High-affinity Antibody-antigen Binding Kinetics Using Four Biosensor Platforms.” Journal of visualized experiments: JoVE 122 (2017), which is incorporated by reference herein in its entirety. Specific illustrative and exemplary embodiments for measuring binding affinity are described in the following. For example, se WO2003056296; Neri, Dario, et al. “Biophysical methods for the determination of antibody-antigen affinities.” Trends in biotechnology 14.12 (1996): 465-470; Leonard, Paul et al. “Measuring protein-protein interactions using Biacore.” Protein Chromatography. Humana Press, 2011. 403-418; and Karlsson, Robert, et al. “Analyzing a kinetic titration series using affinity biosensors.” Analytical biochemistry 349.1 (2006): 136-147. each of which are incorporated by reference herein in there entireties.

An “affinity matured” antibody can be, for example, an antibody with one or more alterations in one or more hypervariable regions (HVRs), compared to a parent antibody which does not possess such alterations, where such alterations can result in an improvement in the affinity of the antibody for antigen.

An “amino acid modification” for example, can be a change in the amino acid sequence of a predetermined amino acid sequence. Exemplary modifications include an amino acid substitution, insertion and/or deletion. The preferred amino acid modification herein is a substitution. An “amino acid modification at” a specified position, e.g. of the Fc region, can refer to the substitution or deletion of the specified residue, or the insertion of at least one amino acid residue adjacent the specified residue. By insertion “adjacent” a specified residue can be, for example, an insertion within one to two residues thereof. The insertion may be N-terminal or C-terminal to the specified residue.

An “amino acid substitution” refers to the replacement of at least one existing amino acid residue in a predetermined amino acid sequence with another different “replacement” amino acid residue. The replacement residue or residues may be “naturally occurring amino acid residues” (i.e. encoded by the genetic code) and selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile); leucine (Leu); lysine (Lys); methionine (Met); phenylalanine (Phe); proline (Pro); serine (Ser); threonine (Thr); tryptophan (Trp); tyrosine (Tyr); and valine (Val). In one embodiment, the replacement residue is not cysteine. Substitution with one or more non-naturally occurring amino acid residues can also refer to an amino acid substitution herein. A “non-naturally occurring amino acid residue” can be, for example, a residue, other than those naturally occurring amino acid residues listed above, which is able to covalently bind adjacent amino acid residues(s) in a polypeptide chain. Non-limiting examples of non-naturally occurring amino acid residues include norleucine, ornithinc, norvaline, homoserine and other amino acid residue analogues such as those described in Ellman, et al., (Meth. Enzym. 202 (1991) 301-336). To generate such non-naturally occurring amino acid residues, the procedures of Noren, et al., (Science 244 (1989) 182 and Ellman, et al., supra) for example. can be used. Briefly. these procedures involve chemically activating a suppressor tRNA with a non-naturally occurring amino acid residue followed by in vitro transcription and translation of the RNA.

An “amino acid insertion” can refer to the incorporation of at least one amino acid into a predetermined amino acid sequence. While the insertion will usually consist of the insertion of one or two amino acid residues, the invention as described herein can utilize larger “peptide insertions”, e.g. an insertion of about three to about five or even up to about ten amino acid residues. The inserted residue(s) may be naturally occurring or non-naturally occurring as described above.

An “amino acid deletion” can refer to the removal of at least one amino acid residue from a predetermined amino acid sequence.

The term “antibody” herein is used in the broadest sense and encompasses various antibody structures, including but not limited to monoclonal antibodies, polyclonal antibodies, multispecific antibodies (e.g., bispecific antibodies), humanized antibodies, and antibody fragments so long as they exhibit the desired antigen-binding activity. Antibodies of the invention include those comprising Fc sequences selected from those described herein. For example, the antibody comprises an Fc variant of a wild-type human IgG Fc region, such as an Fc variant having amino acid substitutions E345K, E430G, L234A. and L235A; or E345K, E430G, S228P and R409K. The residues are numbered according to the EU index of Kabat. In embodiments of the invention, either intact antibody, antibody derivative, or fragment thereof (e.g., antigen binding fragment) can be used. That is, for example, intact antibody, a Fab fragment, an F(ab)2 fragment, a minibody, or a bispecific whole antibody can be used in aspects of the invention, such as to enhance cellular signaling and/or induce receptor clustering.

Toxins can be bound to the antibodies or antibody fragments described herein. Such toxins can include radioisotopes, biological toxins, boronated dendrimers, and immunoliposomes (Chow et al. Adv. Exp. Biol. Med. 746:121-41. 2012)). Toxins can be conjugated to the antibody or antibody fragment using methods well known in the art (Chow et al. Adv. Exp. Biol. Med. 746:121-41 (2012)). Combinations of the antibodies, or fragments or derivatives thereof, disclosed herein can also be used in the methods of the invention.

The term “antibody variant” as used herein refers to, for example, a variant of a wildtype antibody, characterized in that an alteration in the amino acid sequence relative to the wildtype antibody occurs in the antibody variant, e.g. introduced by mutations a specific amino acid residues in the wildtype antibody. For example, the antibody variant can comprise amino acid substitutions in the Fc region that enhance cellular signaling and/or induce receptor clustering. Such substitutions include those described herein, such as E345K, E430G, L234A, and L235A; or E345K, E430G, S228P and R409K in the Fc of human IgG. The residues are numbered according to the EU index of Kabat.

The term “antibody effector function(s),” or “effector function” as used herein can refer to a function contributed by an Fc effector domain(s) of an IgG (e.g., the Fc region of an immunoglobulin). Such function can be effected by, for example, binding of an Fc effector domain(s) to an Fc receptor on an immune cell with phagocytic or lytic activity or by binding of an Fc effector domain(s) to components of the complement system. Typical effector functions are ADCC, ADCP and CDC.

An “antibody fragment” can be a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

An “antibody that binds to the same epitope” as a reference antibody can be, for example, an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

“Antibody-dependent cell-mediated cytotoxicity” and “ADCC” refer to, for example, a cell-mediated reaction in which nonspecific cytotoxic cells that express FcRs (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch, and Kinet, Annu. Rev. Immunol 9 (1991) 457-492.

“Antibody-dependent cellular phagocytosis” and “ADCP” for example, are a process by which antibody-coated cells are internalized, either in whole or in part, by phagocytic immune cells (e.g., macrophages, neutrophils and dendritic cells) that bind to an immunoglobulin Fc region.

“Binding domain,” for example, can be the region of a polypeptide that binds to another molecule. In the case of an FcR, the binding domain can comprise a portion of a polypeptide chain thereof (e.g. the a chain thereof) which is responsible for binding an Fc region. One useful binding domain is the extracellular domain of an FcR a chain.

For example, “binding” to an Fc receptor can be the binding of the antibody to a Fc receptor in a BIAcore.®. assay for example (Pharmacia Biosensor AB, Uppsala, Sweden).

In the BIAcore.®. assay the Fc receptor is bound to a surface and binding of the variant. e.g. the antibody variant to which mutations have been introduced, is measured by Surface Plasmon Resonance (SPR). See, for example, Rich, Rebecca L., and David G. Myszka. “Advances in surface plasmon resonance biosensor analysis.” Current opinion in biotechnology 11.1 (2000): 54-61; and Rich, Rebecca L.; Rich, Rebecca L., and David G. Myszka. “Spying on HIV with SPR.” Trends in microbiology 11.3 (2003): 124-133; McDonnell, James M. “Surface plasmon resonance: towards an understanding of the mechanisms of biological molecular recognition.” Current opinion in chemical biology 5.5 (2001): 572-577; and David G. Myszka. “BIACORE J: a new platform for routine biomolecular interaction analysis.” Journal of Molecular Recognition 14.4 (2001): 223-228, each of which are incorporated by reference herein in their entireties. The affinity of the binding can be defined by the terms kₐ (rate constant for the association of the antibody from the antibody/Fc receptor complex), k_{d} (dissociation constant), and K_{D} (kd/ka).

Alternatively, for example, the binding signal of a SPR sensogram can be compared directly to the response signal of a reference, with respect to the resonance signal height and the dissociation behaviors.

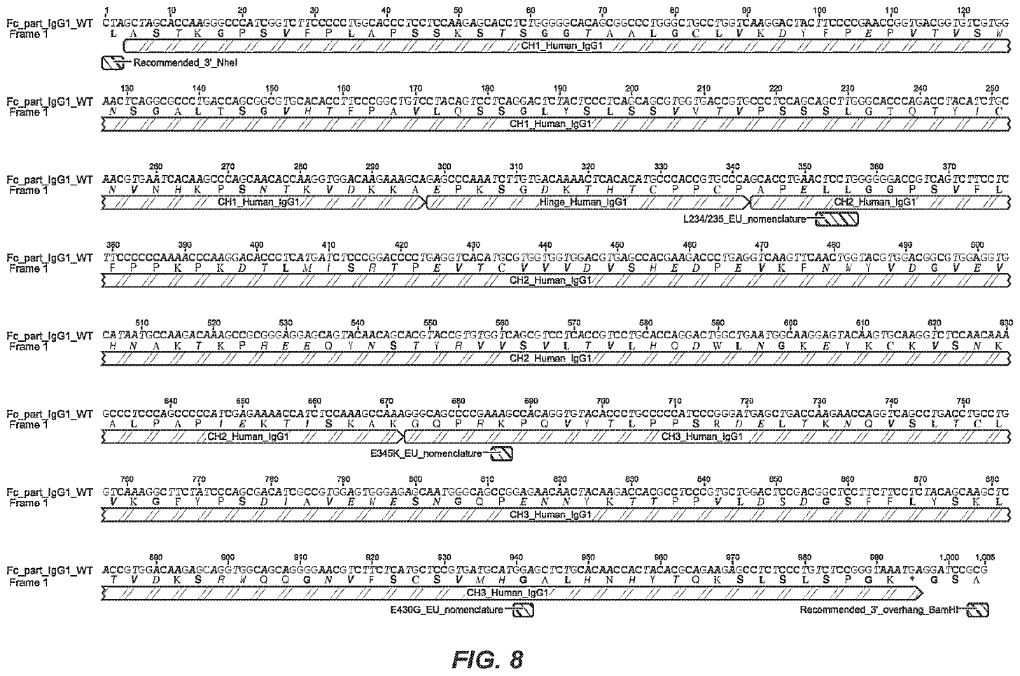

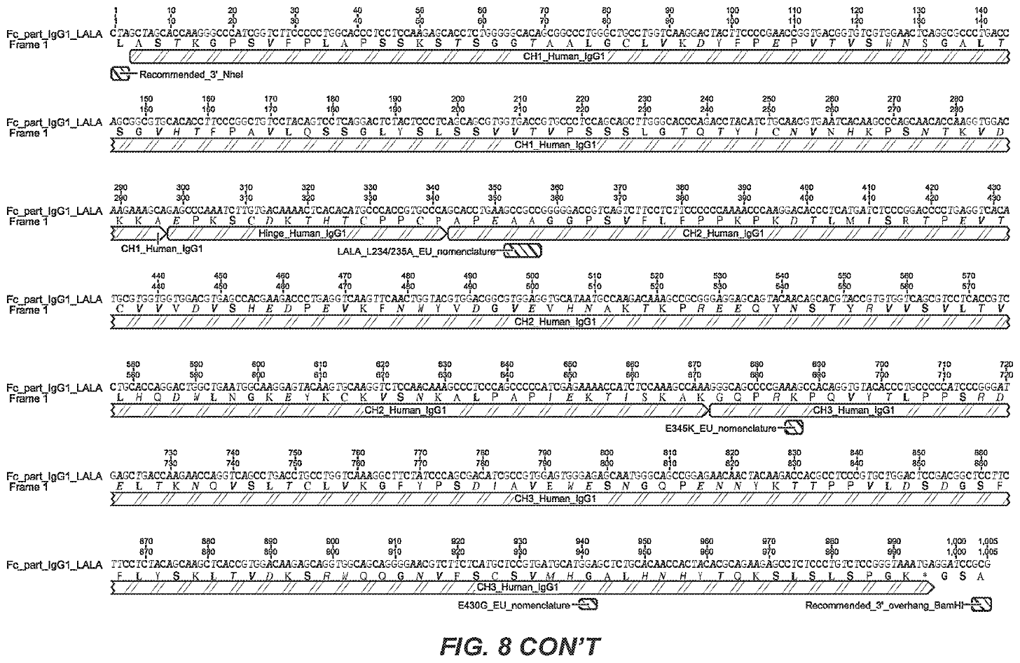

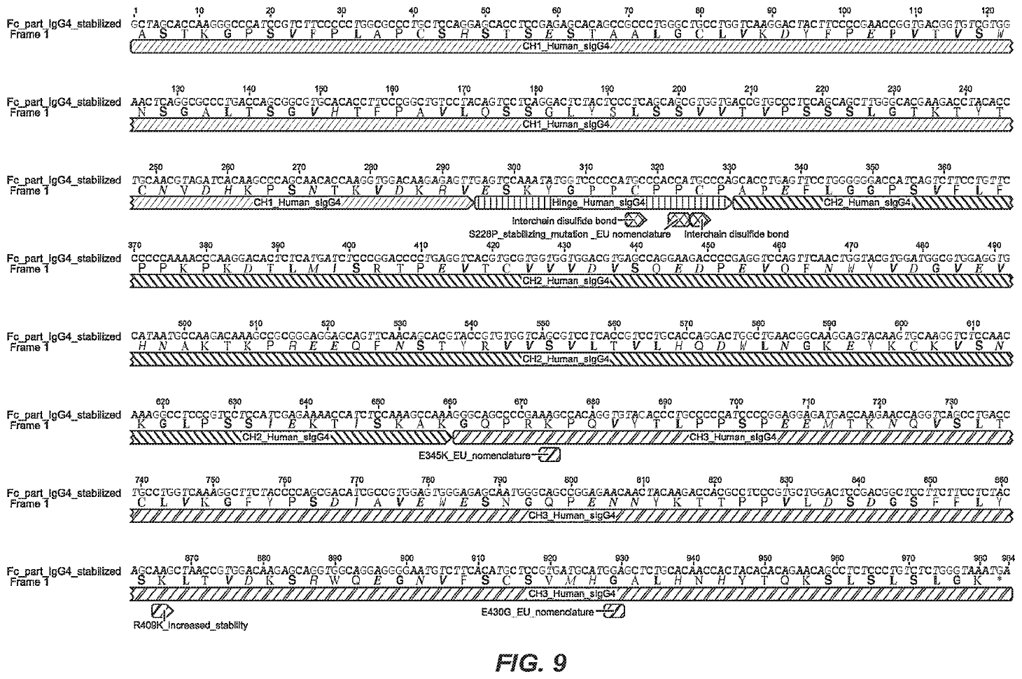

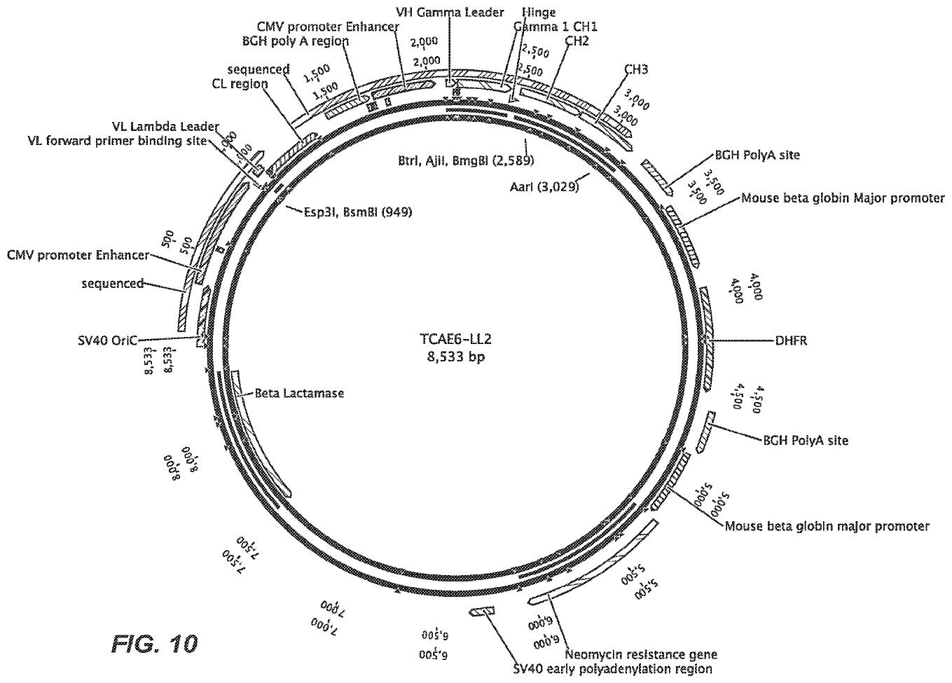

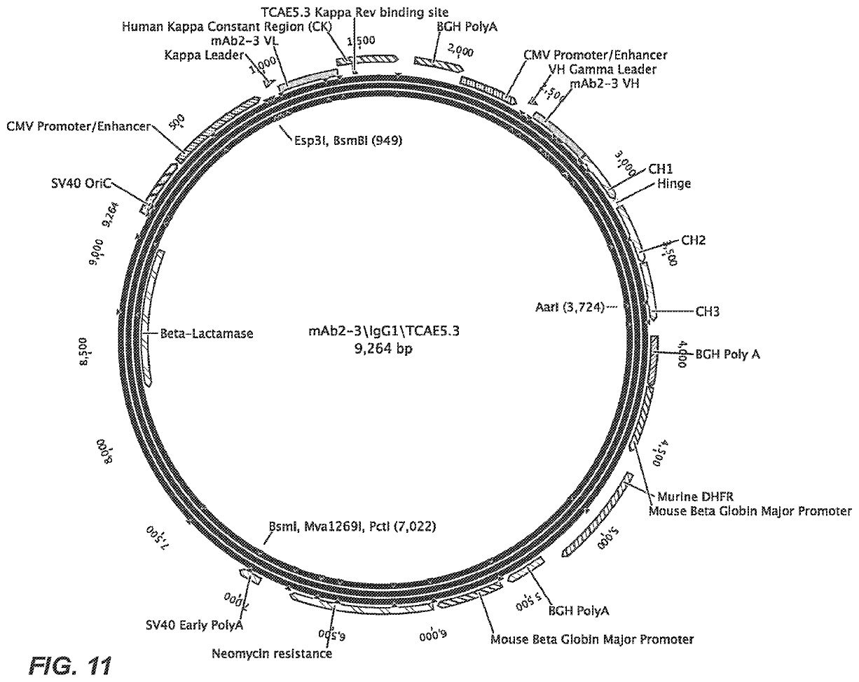

The “CH2 domain” of a human IgG Fc region (also referred to as “Cγ2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain (Burton, Molec. Immunol. 22 (1985) 161-206). In one embodiment, FIGS. 8, 9 and 11 illustrate the CH domains of IgG1, IgG2, and IgG4, respectively.

The “CH3 domain” comprises the stretch of residues C-terminal to a CH2 domain in an Fc region (i.e. from about amino acid residue 341 to about amino acid residue 447 of an IgG). In one embodiment. FIGS. 8, 9 and 11 illustrate the CH domains of IgG1, IgG2, and IgG4, respectively.

“Cancer” and “cancerous” refer to or describe. for example, the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, small-cell lung cancer, non-small cell lung cancer. adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

As used herein, the expressions “cell,” “cell line,” and “cell culture” are used interchangeably and all such designations include progeny. Thus, the words “transformants” and “transformed cells” include the primary subject cell and cultures derived there from without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. For example, see Vidarsson et al. “IgG subclasses and allotypes: from structure to effector functions.” Frontiers in immunology 5 (2014): 520, and Spiegelberg, Hans L. “Biological Activities of Immunoglobulins of Different Classes and Subclasses1.” Advances in immunology. Vol. 19. Academic Press, 1974. 259-294. the entirety of each of which are incorporated by reference herein in their entireties. The heavy chain constant domains that correspond to the different classes of immunoglobulins are called α, δ, ϵ, γ, and μ, respectively.

For example, “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents discussed herein.

“Complement-dependent cytotoxicity” or CDC refers, for example, to a mechanism for inducing cell death in which an Fc effector domain(s) of a target-bound antibody activates a series of enzymatic reactions culminating in the formation of holes in the target cell membrane. Antigen-antibody complexes such as those on antibody-coated target cells bind and activate complement component Clq which in turn activates the complement cascade leading to target cell death. Activation of complement may also result in deposition of complement components on the target cell surface that facilitate ADCC by binding complement receptors (e.g., CR3) on leukocytes.

A “disorder” can be any condition that would benefit from treatment with a polypeptide, like antibodies comprising an Fc variant. This includes chronic and acute disorders or diseases including those pathological conditions which predispose the mammal to the disorder in question. In one embodiment, the disorder is cancer.

“Effector functions,” for example, refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis (ADCP); down regulation of cell surface receptors (e.g. B cell receptor); and B cell activation.

A “reduced effector function” as used herein can refer to a reduction of a specific effector function, like for example ADCC or CDC, in comparison to a control (for example a polypeptide with a wildtype Fc region), by at least 20% and a “strongly reduced effector function” as used herein can refer to a reduction of a specific effector function, like for example ADCC or CDC, in comparison to a control, by at least 50%.

An “effective amount” of an agent, e.g., a pharmaceutical formulation, refers to an amount effective, at dosages and for periods of time necessary, to achieve the desired therapeutic or prophylactic result.

“Fc region,” for example, refers to a C-terminal region of an immunoglobulin heavy chain that contains at least a portion of the constant region. The term can include native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat, et al., Sequences of Proteins of Immunological Interest, 5th Ed. Public Health Service, National Institutes of Health, Bethesda, Md. (1991).

A “variant Fc region” comprises an amino acid sequence which differs from that of a “native” or “wildtype” sequence Fc region by virtue of at least one “amino acid modification” as described herein. In one embodiment, the variant Fc region has at least one amino acid substitution compared to a native sequence Fc region or to the Fc region of a parent polypeptide, e.g. from about one to about ten amino acid substitutions. In one embodiment, the variant Fc region has about one to about five amino acid substitutions in a native sequence Fc region or in the Fc region of the parent polypeptide. The variant Fc region herein can possess at least about 80% homology with a native sequence Fc region and/or with an Fc region of a parent polypeptide, and possess at least about 90% homology therewith, possess at least about 95% homology therewith, possess at least about 96% homology therewith, possess at least about 97% homology therewith, possess at least about 98% homology therewith, or possess at least about 99% homology therewith.

An “Fc-variant” as used herein refers to a polypeptide comprising a modification in an Fc domain. The Fc variants of the present invention are defined according to the amino acid modifications that compose them. Thus, for example, P329G is an Fc variant with the substitution of proline with glycine at position 329 relative to the parent Fc polypeptide, wherein the numbering is according to the EU index. The identity of the wildtype amino acid may be unspecified, in which case the aforementioned variant is referred to as P329G. For all positions discussed in the present invention, numbering is according to the EU index. The EU index or EU index as in Kabat or EU numbering scheme refers to the numbering of the EU antibody (Edelman, et al., Proc Natl Acad Sci USA 63 (1969) 78-85, hereby entirely incorporated by reference.) The modification can be an addition, deletion, or substitution. Substitutions can include naturally occurring amino acids and non-naturally occurring amino acids. Variants may comprise non-natural amino acids. Examples include U.S. Pat. No. 6,586,207; WO 98/48032; WO 03/073238; US 2004/0214988 A1; WO 05/35727 A2; WO 05/74524 A2; Chin, J. W., et al., Journal of the American Chemical Society 124 (2002) 9026-9027; Chin, J. W. and Schultz, P. G., ChemBioChem 11 (2002) 1135-1137; Chin, J. W., et al., PICAS United States of America 99 (2002) 11020-11024; and, Wang. L., and Schultz, P. G., Chem. (2002) 1-10, all entirely incorporated by reference.

“Fc region-containing polypeptide” refers to a polypeptide, such as an antibody or immunoadhesin (see descriptions herein), which comprises an Fc region.

“Fc receptor” or “FcR,” for example, are used to describe a receptor that binds to the Fc region of an antibody. An exemplary FER is a native sequence human FcR. Moreover, another exemplary FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review in Daeron, M., Annu. Rev. Immunol. 15 (1997) 203-234)). FcRs are reviewed in Ravetch, and Kinet, Annu. Rev. Immunol 9 (1991) 457-492; Capel. et al., Immunomethods 4 (1994) 25-34; and de Haas, et al., J. Lab. Clin. Med. 126 (1995) 330-41. Other FcRs, including those to be identified in the future, are encompassed by the term “FcR” herein. The term also includes the neonatal receptor, FcRn, which is responsible for the transfer of maternal IgGs to the fetus (Guyer, et al., J. Immunol. 117 (1976) 587 and Kim, et al., J. Immunol. 24 (1994) 249).

For example, an “IgG Fc ligand” can be a molecule, for example a polypeptide, from any organism that binds to the Fc region of an IgG antibody to form an Fc/Fc ligand complex, Fc ligands include but are not limited to FcγRs, FcRn, Clq, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis, et al., Immunological Reviews 190 (2002) 123-136, entirely incorporated by reference). Fc ligands may include undiscovered molecules that bind Fc. Particular IgG Fc ligands are FcRn and Fc gamma receptors. In one embodiment, “Fc ligand” can be a molecule, for example a polypeptide, from any organism that binds to the Fc region of an antibody to form an Fc/Fc ligand complex.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIA, FcγRIB, and FcγRIC; FcγRII (CD32), including isoforms FcγRIIA (including allotypes H131 and R131), FcγRIIB (including FcγRIIB-1 and FcγRIIB-2), and FcγRIIc; and FcγRIII (CD16), including isoforms FcγRIIIA (including allotypes V158 and F158) and FcγRIIIb (including allotypes FcγRIIB-NA1 and FcγRIIB-NA2) (Jefferis, et al., Immunol Lett 82 (2002) 57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

“FcRn” or “neonatal Fc Receptor,” for example, can be a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless other wise noted herein, FcRn or an FcRn protein refers to the complex of FcRn heavy chain with beta-2-microglobulin.

For example, “wildtype or parent polypeptide” can be an unmodified polypeptide that is subsequently modified to generate a variant. The wildtype polypeptide may be a naturally occurring polypeptide, or a variant or engineered version of a naturally occurring polypeptide. Wildtype polypeptide may refer to the polypeptide itself, compositions that comprise the parent polypeptide, or the amino acid sequence that encodes it. Accordingly, “wildtype immunoglobulin” refers to an unmodified immunoglobulin polypeptide that is modified to generate a variant, and “wildtype antibody” refers to an unmodified antibody that is modified to generate a variant antibody. It should be noted that “wildtype antibody” includes known commercial, recombinantly produced antibodies as described herein.

A “fragment crystallizable (Fc) polypeptide” is the portion of an antibody molecule that interacts with effector molecules and cells. It comprises the C-terminal portions of the immunoglobulin heavy chains.

“Framework” or “FR,” for example, refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

“Full length antibody,” “intact antibody,” and “whole antibody” are used herein interchangeably to refer to an antibody having a structure substantially similar to a native antibody structure or having heavy chains that contain an Fc region as defined herein.

A “functional Fc region” possesses an “effector function” of a native sequence Fc region. Exemplary “effector functions” include Clq binding; complement dependent cytotoxicity; Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays as herein disclosed, for example.

“Hinge region” is generally referred to the stretch of amino acids from Glu216 to Pro230 of human IgG1 (Burton, Molec. Immunol. 22 (1985) 161-206). Hinge regions of other IgG isotypes may be aligned with the IgG1 sequence by placing the first and last cysteine residues forming inter-heavy chain S—S bonds in the same positions.

The “lower hinge region” of an Fc region corresponds to, for example, the stretch of residues immediately C-terminal to the hinge region, i.e. residues 233 to 239 of the Fc region.

“Homology” refers to, for example, as the percentage of residues in the amino acid sequence variant that are identical after aligning the sequences and introducing gaps, if necessary, to achieve the maximum percent homology. Methods and computer programs for the alignment are well known in the art. One such computer program is “Align 2”, authored by Genentech, Inc., which was filed with user documentation in the United States Copyright Office, Washington, D.C. 20559, on Dec. 10, 1991.

The terms “host cell,” “host cell line.” and “host cell culture” are used interchangeably and refer to cells into which exogenous nucleic acid has been introduced, including the progeny of such cells. Host cells include “transformants” and “transformed cells,” which include the primary transformed cell and progeny derived there from without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. A human antibody specifically excludes a humanized antibody comprising non-human antigen-binding residues.

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g. from blood or PBMCs as described herein.

A “humanized” antibody can refer to, for example, a chimeric antibody comprising amino acid residues from non-human HVRs and amino acid residues from human FRs. In certain embodiments, a humanized antibody can comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally can comprise at least a portion of an antibody constant region derived from a human antibody. A “humanized form” of an antibody, e.g., a non-human antibody, refers to an antibody that has undergone humanization. For example, “chimeric” antibody refers to an antibody in which a portion of the heavy and/or light chain is derived from a particular source or species, while the remainder of the heavy and/or light chain is derived from a different source or species.

“Hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3) (Chothia, and Lesk. J. Mol. Biol. 196 (1987) 901-917). Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3 (Kabat. et al., Sequences of Proteins of Immunological Interest, 5th Ed. Public Health Service, National Institutes of Health, Bethesda, Md. (1991)). With the exception of CDR1 in VH, CDRs generally comprise the amino acid residues that form the hypervariable loops, CDRs also comprise “specificity determining residues,” or “SDRs,” which are residues that contact antigen, SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3 (See Almagro, and Fransson, Front. Biosci. 13 (2008) 1619-1633). Unless otherwise indicated, HVR residues and other residues in the variable domain (e.g., FR residues) are numbered herein according to Kabat et al., supra.

“Immune complex” refers to the relatively stable structure which forms when at least one target molecule and at least one heterologous Fc region-containing polypeptide bind to one another forming a larger molecular weight complex. Examples of immune complexes are antigen-antibody aggregates and target molecule-immunoadhesin aggregates. The term “immune complex” as used herein, unless indicated otherwise, refers to an ex vivo complex (i.e. other than the form or setting in which it may be found in nature). However, the immune complex may be administered to a mammal, e.g. to evaluate clearance of the immune complex in the mammal.

An “immunoconjugate” is an antibody conjugated to one or more heterologous molecule(s), including but not limited to a cytotoxic agent.

An “individual” or “subject” can be a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

The term “subject” or “patient” can refer to any organism to which aspects of the invention can be administered, e.g., for experimental, diagnostic, prophylactic, and/or therapeutic purposes. Typical subjects to which compounds of the present disclosure may be administered will be mammals. particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to a subject noted above or another organism that is alive. The term “living subject” refers to the entire subject or organism and not just a part excised (e.g., a liver or other organ) from the living subject.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### ADCC Assays

We performed ADCC assays using a reporter system from Promega. A pool of CHO-GITR cells were sorted to attain a cell population with a purity of >99% GITR+ cells. The cells were plated at 15k cells/well and incubated with various concentrations of the different aGITR antibodies. Promega ADCC Bioassay Effector Cells were added at a 5:1 E:T ratio and the plates were incubated for 6 hours at 37 C., 5% CO2. Following incubation, Bio-Glo Lucifierase Assay reagent was added and the luminescent signal was detected using BMG PolarStart Multilabel plate reader. The data illustrate that only the IgG1 WT monomer and hexamer constructs showed significant ADCC activity as expected. It is also interesting to note that hexamerization appears to lower the magnitude of ADCC in WT IgG1. Negative control IgG showed no specific ADCC activities.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

Those skilled in the art will recognize, or be able to ascertain, using no more than routine experimentation, numerous equivalents to the specific substances and procedures described herein. Such equivalents are considered to be within the scope of this invention, and are covered by the following claims.

1. An engineered polypeptide comprising an IgG4 Fc variant of a wild-type human IgG Fc region, wherein the Fc variant comprises amino acid substitutions to promote hexamerization at residue positions 345 and 430, wherein the glutamate (E) at residue position 345 is substituted with lysine (K); and wherein the glutamate (E) at residue position 430 is substituted with glycine (G); and
amino acid substitutions to promote stabilization at residue positions 228 and 409, wherein the serine(S) at residue position 228 is substituted with proline (P), and the arginine (R) at residue position 409 is substituted with lysine (K), and wherein the amino acid residues are numbered according to the EU index of Kabat.

2. (canceled)

3. The polypeptide of claim 1, wherein the amino acid at residue position 234 according to the EU index of Kabat is substituted with alanine (A).

4. The polypeptide of claim 1, wherein the amino acid at residue position 235 according to the EU index of Kabat is substituted with alanine (A).

5. (canceled)

6. (canceled)

7. (canceled)

8. The polypeptide of claim 1, wherein serine(S) at residue position 440 according to the EU index of Kabat is substituted with tryptophan (W).

9. (canceled)

10. (canceled)

11. The polypeptide of claim 1, wherein the polypeptide exhibits a reduced affinity to one or more of human Fc receptors compared to the polypeptide comprising the wildtype IgG Fc region.

12. The polypeptide of claim 11, wherein the polypeptide further exhibits increased receptor clustering compared to the polypeptide comprising the wildtype IgG Fc region.

13. (canceled)

14. The polypeptide of claim 1, wherein the polypeptide is an antibody or an Fc fusion protein.

15. The polypeptide of claim 14, wherein the antibody is a monospecific antibody, a bispecific antibody, or a multispecific antibody.

16. The polypeptide according to claim 1, wherein the polypeptide is conjugated to a drug, a toxin, a radiolabel, or a combination thereof.

17. The polypeptide according to claim 1, wherein the polypeptide is an antibody specific for BCMA, CAIX, CCR4, PD-L1, PD-L2, PD1, Glucocorticoid-Induced Tumor Necrosis Factor Receptors (GITR), TIGIT, Severe acute respiratory syndrome (SARS), Middle East Respiratory Syndrome (MERS), influenza or flavivirus.

18. The polypeptide according to claim 1, wherein the polypeptide is an antibody specific for Glucocorticoid-Induced Tumor Necrosis Factor Receptors (GITR).

19. The polypeptide according to claim 1, wherein the polypeptide is an antibody specific for CCR4.

20. An engineered polypeptide comprising an Fc variant of a wild-type human IgG Fc region, wherein the Fc variant comprises an amino acid sequence comprising at least 90% identity to SEQ ID NO: 4, and wherein an amino acid substitution occurs at X₁, X₂, X₃, X₄, X₅, X₆, X₇, or a combination thereof.

21.-27. (canceled)

28. An engineered polypeptide comprising an Fc variant of a wild-type human IgG Fc region, wherein the Fc variant comprises an amino acid sequence comprising at least 90% identity to SEQ ID NO: 5, and wherein an amino acid substitution occurs at X₁, X₂, X₃, X₄, X₅, X₆, or a combination thereof.

29.-34. (canceled)

35. An engineered polypeptide comprising an Fc variant of a wild-type human IgG Fc region, wherein the Fc variant comprises an amino acid sequence comprising at least 90% identity to SEQ ID NO: 6, and wherein an amino acid substitution occurs at X₁, X₂, X₃, X₄, X₅, X₆, X₇, or a combination thereof.

36.-42. (canceled)

43. A recombinant GITR antibody, wherein the antibody comprises the variable region amino acid sequences disclosed in Table 1B and the variant Fc region amino acid sequences disclosed in Table 3B (SEQ ID NOS: 18, 19, 21, 22, 24), Table 4B (SEQ ID NOS: 18, 19, 20, 22, 26), Table 5B (SEQ ID NOS: 18, 19, 22, 29, and 30), or Table 6B (SEQ ID NOS: 36, 37, 38, 40, and 42).

44. A recombinant CCR4 antibody, wherein the antibody comprises the variable region amino acid sequences disclosed in Table 1B and the variant Fc region amino acid sequences disclosed in Table 3B (SEQ ID NOS: 18, 19, 21, 24), Table 4B (SEQ ID NOS: 18, 19, 20, 26), Table 5B (SEQ ID NOS: 18, 19, 29, and 30), or Table 6B (SEQ ID NOS: 36, 37, 38, and 42).

45. A method of treating a tumor in a subject, the method comprising administering to the subject the antibody of claim 1.

46. A method of treating a blood-based cancer, the method comprising administering to a subject the antibody of claim 1.

47. The method of claim 46, wherein the blood-based cancer is a lymphoma or a leukemia.

48. A method of enhancing cellular signaling of a cell, the method comprising: contacting the cell with an antibody that binds a ligand onto the cell, and wherein the antibody comprises the antibody of claim 1.

49. (canceled)

50. (canceled)

51. A method of inducing receptor clustering of a cell, the method comprising: contacting the cell with an antibody that binds a ligand onto the cell, and wherein the antibody comprises the antibody of claim 1.

52. (canceled)

53. (canceled)

54. The method of claim 45, wherein tumor is a solid tumor or liquid tumor.


SEQUENCE LISTING (77 sequences)

SEQ ID NO: 1 - protein, 330 aa, Homo sapiens [sequence omitted]

SEQ ID NO: 2 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 3 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 330 aa, synthetic construct
  VARIANT 228: note = Lys, Gln, Arg or Tyr
  VARIANT 313: note = Gly, Ser, Phe or Thr
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRXPQVYTLPPSRDELTKNQVSLTCLVKGFYPSDIAVEWESNGQPENNYKTTPPVLDSDGSFFLYSRLTVDKSRWQQGNVFSCSVMHXALHNHYTQKWLSLSPGK

SEQ ID NO: 5 - protein, 326 aa, synthetic construct
  VARIANT 224: note = Lys, Gln, Arg or Tyr
  VARIANT 309: note = Gly, Ser, Phe or Thr
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPRXPQVYTLPPSREEMTKNQVSLTCLVKGFYPSDISVEWESNGQPENNYKTTPPMLDSDGSFFLYSRLTVDKSRWQQGNVFSCSVMHXALHNHYTQKWLSLSPGK

SEQ ID NO: 6 - protein, 327 aa, synthetic construct
  VARIANT 225: note = Lys, Gln, Arg or Tyr
  VARIANT 310: note = Gly, Ser, Phe or Thr
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPRXPQVYTLPPSQEEMTKNQVSLTCLVKGFYPSDIAVEWESNGQPENNYKTTPPVLDSDGSFFLYSKLTVDKSRWQEGNVFSCSVMHXALHNHYTQKWLSLSLGK

SEQ ID NO: 7 - DNA, 355 bp, synthetic construct
  source 1..355: mol_type = other DNA
caggtgcagctggtgcagtctgggggaggcttggtacagcctggggggtccctgagactctcctgtgcagcctctggattcacctttagcagccatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaatcggtacggcggatgcttttgatatctggggccaagggaccacggtcaccgtctcctcag

SEQ ID NO: 8 - DNA, 331 bp, synthetic construct
  source 1..331: mol_type = other DNA
cagtctgccctgactcagccaccctcagtgtctgggacccccggacagagggtcaccatctcttgttctggaggcgtccccaacatcggaagtaatcctgtaaactggtacctccaccgcccaggaacggcccccaaactcctcatctataatagcaatcagtggccctcaggggtccctgaccgattttctggctccaggtctggcacctcagcctccctggccatcagtgggctccagtctgaggatgaggctgattattactgtgcagcatgggatgacagcctggatggtctggttttcggcggagggaccaagttgaccgtcctag

SEQ ID NO: 9 - protein, 118 aa, synthetic construct
QVQLVQSGGGLVQPGGSLRLSCAASGFTFSSHAMSWVRQAPGKGLEWVSAISGSGGSTYYADSVKGRFTISRDNSKNTLYLQMNSLRAEDTAVYYCAKIGTADAFDIWGQGTTVTVSS

SEQ ID NO: 10 - protein, 110 aa, synthetic construct
QSALTQPPSVSGTPGQRVTISCSGGVPNIGSNPVNWYLHRPGTAPKLLIYNSNQWPSGVPDRFSGSRSGTSASLAISGLQSEDEADYYCAAWDDSLDGLVFGGGTKLTVL

SEQ ID NO: 11 - protein, 118 aa, synthetic construct
QVQLVQSGAEVKKPGASVKVSCKASGYTFASAWMHWMRQAPGQGLEWIGWINPGNVNTKYNEKFKGRATLTVDTSTNTAYMELSSLRSEDTAVYYCARSTYYRPLDYWGQGTLVTVSS

SEQ ID NO: 12 - protein, 112 aa, synthetic construct
DIVMTQSPDSLAVSLGERATINCKSSQSILYSSNQKNYLAWYQQKPGQSPKLLIYWASTRESGVPDRFSGSGSGTDFTLTISSLQAEDVAVYYCHQYMSSYTFGQGTKLEIK

SEQ ID NO: 13 - DNA, 285 bp, Homo sapiens
  source 1..285: mol_type = unassigned DNA
accaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaa

SEQ ID NO: 14 - DNA, 48 bp, Homo sapiens
  source 1..48: mol_type = unassigned DNA
gcagagcccaaatcttgtgacaaaactcacacatgcccaccgtgccca

SEQ ID NO: 15 - DNA, 330 bp, Homo sapiens
  source 1..330: mol_type = unassigned DNA
gcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaa

SEQ ID NO: 16 - DNA, 324 bp, Homo sapiens
  source 1..324: mol_type = unassigned DNA
gggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 17 - DNA, 321 bp, Homo sapiens
  source 1..321: mol_type = unassigned DNA
ggtcagcccaaggctgccccctcggtcactctgttcccgccctcctctgaggagcttcaagccaacaaggccacactggtgtgtctcataagtgacttctacccgggagccgtgacagtggcctggaaggcagatggcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatga

SEQ ID NO: 18 - protein, 97 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKK

SEQ ID NO: 19 - protein, 16 aa, Homo sapiens
AEPKSCDKTHTCPPCP

SEQ ID NO: 20 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 21 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 106 aa, Homo sapiens
GQPKAAPSVTLFPPSSEELQANKATLVCLISDFYPGAVTVAWKADGSPVKAGVETTTPSKQSNNKYAASSYLSLTPEQWKSHRSYSCQVTHEGSTVEKTVAPTECS

SEQ ID NO: 23 - DNA, 330 bp, synthetic construct
  source 1..330: mol_type = other DNA
gcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaa

SEQ ID NO: 24 - protein, 110 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 25 - DNA, 324 bp, synthetic construct
  source 1..324: mol_type = other DNA
gggcagccccgaaagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatggagctctgcacaaccactacacgcagaagagcctctccctgtctccgggtaaatga

SEQ ID NO: 26 - protein, 107 aa, synthetic construct
GQPRKPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 27 - DNA, 330 bp, synthetic construct; identical to SEQ ID NO: 23
  source 1..330: mol_type = other DNA

SEQ ID NO: 28 - DNA, 324 bp, synthetic construct; identical to SEQ ID NO: 25
  source 1..324: mol_type = other DNA

SEQ ID NO: 29 - protein, 110 aa, synthetic construct; identical to SEQ ID NO: 24

SEQ ID NO: 30 - protein, 107 aa, synthetic construct; identical to SEQ ID NO: 26

SEQ ID NO: 31 - DNA, 294 bp, synthetic construct
  source 1..294: mol_type = other DNA
gctagcaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagtt

SEQ ID NO: 32 - DNA, 36 bp, synthetic construct
  source 1..36: mol_type = other DNA
gagtccaaatatggtcccccatgcccaccatgccca

SEQ ID NO: 33 - DNA, 330 bp, synthetic construct
  source 1..330: mol_type = other DNA
gcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaa

SEQ ID NO: 34 - DNA, 324 bp, synthetic construct
  source 1..324: mol_type = other DNA
gggcagccccgagagccacaggtgtacaccctgcccccatccccggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctaaccgtggacaagagcaggtggcaggaggggaatgtcttctcatgctccgtgatgcatgaggctctgcacaaccactacacacagaagagcctctccctgtctctgggtaaatga

SEQ ID NO: 35 - DNA, 321 bp, synthetic construct; identical to SEQ ID NO: 17
  source 1..321: mol_type = other DNA

SEQ ID NO: 36 - protein, 98 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 37 - protein, 12 aa, synthetic construct
ESKYGPPCPPCP

SEQ ID NO: 38 - protein, 110 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 39 - protein, 107 aa, synthetic construct
GQPREPQVYTLPPSPEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 40 - protein, 106 aa, synthetic construct; identical to SEQ ID NO: 22

SEQ ID NO: 41 - DNA, 324 bp, synthetic construct
  source 1..324: mol_type = other DNA
gggcagccccgaaagccacaggtgtacaccctgcccccatccccggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctaaccgtggacaagagcaggtggcaggaggggaatgtcttctcatgctccgtgatgcatggagctctgcacaaccactacacacagaagagcctctccctgtctctgggtaaatga

SEQ ID NO: 42 - protein, 107 aa, synthetic construct
GQPRKPQVYTLPPSPEEMTKNQVSLTCLVKGFYPSDIAVEWESNGQPENNYKTTPPVLDSDGSFFLYSKLTVDKSRWQEGNVFSCSVMHGALHNHYTQKSLSLSLGK

SEQ ID NO: 43 - DNA, 324 bp, synthetic construct
  source 1..324: mol_type = other DNA
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 44 - protein, 107 aa, synthetic construct [sequence omitted]

SEQ ID NO: 45 - protein, 25 aa, synthetic construct
QVQLVQSGGGLVQPGGSLRLSCAAS

SEQ ID NO: 46 - protein, 8 aa, synthetic construct
GFTFSSHA

SEQ ID NO: 47 - protein, 17 aa, synthetic construct
MSWVRQAPGKGLEWVSA

SEQ ID NO: 48 - protein, 8 aa, synthetic construct
ISGSGGST

SEQ ID NO: 49 - protein, 38 aa, synthetic construct
YYADSVKGRFTISRDNSKNTLYLQMNSLRAEDTAVYYC

SEQ ID NO: 50 - protein, 11 aa, synthetic construct
AKIGTADAFDI

SEQ ID NO: 51 - protein, 11 aa, synthetic construct
WGQGTTVTVSS

SEQ ID NO: 52 - protein, 25 aa, synthetic construct
QSALTQPPSVSGTPGQRVTISCSGG

SEQ ID NO: 53 - protein, 8 aa, synthetic construct
VPNIGSNP

SEQ ID NO: 54 - protein, 17 aa, synthetic construct
VNWYLHRPGTAPKLLIY

SEQ ID NO: 55 - protein, 36 aa, synthetic construct
QWPSGVPDRFSGSRSGTSASLAISGLQSEDEADYYC

SEQ ID NO: 56 - protein, 11 aa, synthetic construct
AAWDDSLDGLV

SEQ ID NO: 57 - protein, 10 aa, synthetic construct
FGGGTKLTVL

SEQ ID NO: 58 - protein, 25 aa, synthetic construct [sequence omitted]

SEQ ID NO: 59 - protein, 8 aa, synthetic construct
GYTFASAW

SEQ ID NO: 60 - protein, 17 aa, synthetic construct
MHWMRQAPGQGLEWIGW

SEQ ID NO: 61 - protein, 8 aa, synthetic construct
INPGNVNT

SEQ ID NO: 62 - protein, 40 aa, synthetic construct
KYNEKFKGRATLTVDTSTNTAYMELSSLRSEDTAVYYCAR

SEQ ID NO: 63 - protein, 9 aa, synthetic construct
STYYRPLDY

SEQ ID NO: 64 - protein, 11 aa, synthetic construct
WGQGTLVTVSS

SEQ ID NO: 65 - protein, 26 aa, synthetic construct
DIVMTQSPDSLAVSLGERATINCKSS

SEQ ID NO: 66 - protein, 12 aa, synthetic construct
QSILYSSNQKNY

SEQ ID NO: 67 - protein, 17 aa, synthetic construct
LAWYQQKPGQSPKLLIY

SEQ ID NO: 68 - protein, 6 aa, synthetic construct
WASTRE

SEQ ID NO: 69 - protein, 33 aa, synthetic construct
SGVPDRFSGSGSGTDFTLTISSLQAEDVAVYYC

SEQ ID NO: 70 - protein, 8 aa, synthetic construct
HQYMSSYT

SEQ ID NO: 71 - protein, 10 aa, synthetic construct
FGQGTKLEIK

SEQ ID NO: 72 - DNA, 1005 bp, Homo sapiens
  source 1..1005: mol_type = unassigned DNA
  CDS 1..996: protein_id = 73; translation = LASTKGPSVFPLAPSSKSTSGGTAALGCLVKDYFPEPVTVSWNS GALTSGVHTFPAVLQSSGLYSLSSVVTVPSSSLGTQTYICNVNHKPSNTKVDKKAEPK SCDKTHTCPPCPAPELLGGPSVFLFPPKPKDTLMISRTPEVTCVVVDVSHEDPEVKFN WYVDGVEVHNAKTKPREEQYNSTYRVVSVLTVLHQDWLNGKEYKCKVSNKALPAPIEK TISKAKGQPRKPQVYTLPPSRDELTKNQVSLTCLVKGFYPSDIAVEWESNGQPENNYK TTPPVLDSDGSFFLYSKLTVDKSRWQQGNVFSCSVMHGALHNHYTQKSLSLSPGK
ctagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgaaagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatggagctctgcacaaccactacacgcagaagagcctctccctgtctccgggtaaatgaggatccgcg

SEQ ID NO: 73 - protein, 331 aa, Homo sapiens
LASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRKPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 74 - DNA, 1005 bp, synthetic construct
  source 1..1005: mol_type = other DNA
  CDS 1..996: protein_id = 75; translation = LASTKGPSVFPLAPSSKSTSGGTAALGCLVKDYFPEPVTVSWNS GALTSGVHTFPAVLQSSGLYSLSSVVTVPSSSLGTQTYICNVNHKPSNTKVDKKAEPK SCDKTHTCPPCPAPEAAGGPSVFLFPPKPKDTLMISRTPEVTCVVVDVSHEDPEVKFN WYVDGVEVHNAKTKPREEQYNSTYRVVSVLTVLHQDWLNGKEYKCKVSNKALPAPIEK TISKAKGQPRKPQVYTLPPSRDELTKNQVSLTCLVKGFYPSDIAVEWESNGQPENNYK TTPPVLDSDGSFFLYSKLTVDKSRWQQGNVFSCSVMHGALHNHYTQKSLSLSPGK
ctagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgaaagccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatggagctctgcacaaccactacacgcagaagagcctctccctgtctccgggtaaatgaggatccgcg

SEQ ID NO: 75 - protein, 331 aa, synthetic construct
LASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRKPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 76 - DNA, 984 bp, synthetic construct
  source 1..984: mol_type = other DNA
  CDS 1..984: protein_id = 77; translation = ASTKGPSVFPLAPCSRSTSESTAALGCLVKDYFPEPVTVSWNSG ALTSGVHTFPAVLQSSGLYSLSSVVTVPSSSLGTKTYTCNVDHKPSNTKVDKRVESKY GPPCPPCPAPEFLGGPSVFLFPPKPKDTLMISRTPEVTCVVVDVSQEDPEVQFNWYVD GVEVHNAKTKPREEQFNSTYRVVSVLTVLHQDWLNGKEYKCKVSNKGLPSSIEKTISK AKGQPRKPQVYTLPPSPEEMTKNQVSLTCLVKGFYPSDIAVEWESNGQPENNYKTTPP VLDSDGSFFLYSKLTVDKSRWQEGNVFSCSVMHGALHNHYTQKSLSLSLGK
gctagcaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgaaagccacaggtgtacaccctgcccccatccccggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaagctaaccgtggacaagagcaggtggcaggaggggaatgtcttctcatgctccgtgatgcatggagctctgcacaaccactacacacagaagagcctctccctgtctctgggtaaatga

SEQ ID NO: 77 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPRKPQVYTLPPSPEEMTKNQVSLTCLVKGFYPSDIAVEWESNGQPENNYKTTPPVLDSDGSFFLYSKLTVDKSRWQEGNVFSCSVMHGALHNHYTQKSLSLSLGK